(19) United States
(12) Patent Application Publication
(10) Pub. No.: US 2026/0315459 A1
(43) Pub. Date: Oct. 15, 2026

(54) CHEMOKINE RECEPTOR CCR1 AS A NEW THERAPEUTIC TARGET TO IMPROVE FLUID RESUSCITATION

(71) Applicant: UNIVERSITY OF SOUTH FLORIDA, Tampa, FL (US)

(72) Inventors: Matthias Majetschak, Tampa, FL (US); Xianlong Gao, Tampa, FL (US); Elizabeth Cook, Tampa, FL (US)

(21) Appl. No.: 19/642,990

(22) Filed: Apr. 9, 2026

Related U.S. Application Data
(60) Provisional application No. 63/785,999, filed on Apr. 9, 2025.

(51) Int. Cl. A61K 31/495; A61K 9/08; A61K 47/02; A61P 7/08

(52) U.S. Cl. CPC A61K 31/495; A61K 9/08; A61K 47/02; A61P 7/08

(57) ABSTRACT

Chemokines and their receptors are known to play roles in inflammatory conditions and responses, including hemorrhagic shock. The chemokine (C-C motif) receptor 1 (CCR1) antagonist BX471 and the CCR2 antagonist INCB3284 were shown to reduce fluid requirements during resuscitation after hemorrhage. Methods of using CCR1 inhibitors alone or with other CCR inhibitors for fluid resuscitation, and to reduce fluid requirements during resuscitation are disclosed.

### CROSS-REFERENCE TO RELATED APPLICATIONS

This application claims the benefit of priority to U.S. Provisional Application No. 63/785,999, filed Apr. 9, 2025, the disclosure of which is incorporated herein by reference in its entirety.

### STATEMENT REGARDING FEDERALLY SPONSORED RESEARCH OR DEVELOPMENT

This invention was made with government support under R35GM152056 and T32GM144274 awarded by the National Institutes of Health. The government has certain rights in the invention.

### TECHNICAL FIELD

This disclosure relates to chemokine receptors (CCR) and more particularly to CCR antagonists.

### BACKGROUND

In the United States, trauma is the leading cause of death up to the age of 50 years and hemorrhagic shock remains a major cause of preventable death after injuries (Rhee P, Holcomb J B, Zangbar B. Evolving Epidemiology of Increasing Trauma Deaths in the United States (2000-2020). Ann Surg. 2025; 281(6):976-81. Epub 2025/02/11. doi: 10.1097/SLA.0000000000006668. PubMed PMID: 39931816; Bernhardt I M, Moore E E, Sauaia A, Cotton B A, Cannon J W, Schreiber M A, et al. Timing of trauma deaths due to uncontrolled bleeding have not changed in three decades: A multicenter study of patients in hemorrhagic shock. Am J Surg. 2025; 250:116510. Epub 2025/07/25. doi: 10.1016/j.amjsurg.2025.116510. PubMed PMID: 40706451). Although prehospital blood transfusion protocols have gained attention in recent years, most trauma patients in the US do not receive blood products in the pre-hospital setting. Moreover, a recent multicenter, allocation concealed, open-label, parallel group, randomized, controlled phase 3 trial did not provide evidence that very early blood product transfusion provides a benefit over crystalloid transfusion in trauma patients (Crombie, N.; Doughty, H. A.; Bishop, J. R. B.; Desai, A.; Dixon, E. F.; Hancox, J. M.; Herbert, M. J.; Leech, C.; Lewis, S. J.; Nash, M. R.; et al. Resuscitation with blood products in patients with trauma-related haemorrhagic shock receiving prehospital care (RePHILL): A multicentre, open-label, randomised, controlled, phase 3 trial. Lancet Haematol. 2022, 9, e250-e261). Furthermore, while pre-hospital fluid resuscitation is essential for the treatment of hemorrhagic shock when blood components are not available, the risks of under- and over-resuscitation are well recognized. The ideal fluid resuscitation strategy and the choice of resuscitation fluid, however, are still a matter of debate (Ramesh G H, Uma J C, Farhath S. Fluid resuscitation in trauma: what are the best strategies and fluids? Int J Emerg Med. 2019; 12(1):38. Epub 2019/12/06. doi: 10.1186/s12245-019-0253-8. PubMed PMID: 31801458; PubMed Central PMCID: PMC6894336). Moreover, drugs that could be used to improve fluid resuscitation after hemorrhagic shock are not available. Such drugs would provide the potential to significantly reduce morbidity and mortality after traumatic-hemorrhagic shock. The compositions and methods disclosed herein address these and other needs.

### SUMMARY

In one example, disclosed herein is a resuscitation fluid comprising a chemokine (C-C motif) receptor 1 (CCR1) inhibitor and an aqueous iso or hypertonic salt solution. In another example, disclosed herein is a method of fluid resuscitation of a patient in need thereof, comprising administering to the patient a resuscitation fluid and a therapeutically effective amount of a CCR1 inhibitor.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

FIGS. 1A-1F provide graphs demonstrating that animals were hemorrhaged to a MAP of 30 mmHg for 30 min, followed by fluid resuscitation for 60 min. At t=30 min, animals were treated with vehicle (open circles, n=5), 0.05 μmol/kg BX471 (light grey squares, n=5) or 0.5 μmol/kg BX471 (dark grey squares, n=4). Arrows indicate the time point of iv vehicle/drug injection. *: p<0.05 vs. vehicle-treated animals. (FIG. 1A) hemorrhage volume in % of total blood volume (% BV). (FIG. 1B) MAP (mmHg). (FIG. 1C) Resuscitation volume (mL/kg). (FIG. 1D) Hematocrit (%). (FIG. 1E) Lactate concentrations (mmol/L). (FIG. 1F) PaO₂, partial pressure of oxygen in arterial blood (mmHg).

FIGS. 2A-2F provide graphs demonstrating that animals were hemorrhaged to a MAP of 30 mmHg for 30 min, followed by fluid resuscitation for 180 min. At t=30 min, animals were treated with vehicle (open circles, n=8) or 0.5 μmol/kg BX471 (dark grey squares,n=7). Arrows indicate the time point of iv vehicle/drug injection. *: p<0.05 vs. vehicle-treated animals. (FIG. 2A) hemorrhage volume in % of total blood volume (% BV). (FIG. 2B) MAP 208 (mmHg). (FIG. 2C) Resuscitation volume (mL/kg). (FIG. 2D) Hematocrit (%). (FIG. 2E) Lactate concentrations (mmol/L). (FIG. 2F) PaO₂, partial pressure of oxygen in arterial blood (mmHg).

FIGS. 3A-3D provide graphs demonstrating the Serum CCR1 ligand concentrations (pg/mg) at baseline (t=0 min) and at the end of the experiment (t=210 min). Light grey bars: vehicle treatment, n=7. Dark grey bars: 0.5 μmol/kg BX471 treatment, n=7. (FIG. 3A) CCL3. (FIG. 3B) CCL4. (FIG. 3C) CCL5 (FIG. 3D) CCL7. *: p<0.05 vs. baseline (t=0 min).

FIGS. 4A-4E provide graphs demonstrating the W/D ratios of small intestine (FIG. 4A), large intestine (FIG. 4B) and lung (FIG. 4C). (FIG. 4D) W/D ratios of the sum of all tissues in % of vehicle. (FIG. 4E) Lung extract MPO activity (RFU/min/mg). Boxes extend from the 25th-75th percentile, and the horizontal line shows the median. Error bars show the range of data (minimum/maximum). Open boxes: vehicle treatment, n=8. Grey boxes: 0.5 μmol/kg BX471 treatment, n=7. The level of statistical significance is indicated.

FIGS. 5A-5C provide graphs demonstrating the inflammation markers in small intestine (FIG. 5A), large intestine (FIG. 5B) and lung (FIG. 5C) tissue extracts, as indicated in the graphs. Data are relative concentrations in % of the OD/mg measured in tissue extracts from vehicle-treated animals. Open bars: vehicle, n=6. Grey bars: 0.5 μmol/kg BX471. *: p<0.05 vs. vehicle.

FIGS. 6A-6I provide graphs demonstrating the effects of BX471 and INCB3284 during resuscitation after hemorrhage. Animals were hemorrhaged to a MAP of 30 mmHg for 30 min followed by 360 min of fluid resuscitation. Animal received either vehicle (open circles, n=12), 0.5 μmol/kg of BX471 (grey squares, n=9), 5 μmol/kg dose of INCB3284 (grey triangles, n=7), or a combination dose of 0.5 μmol/kg of BX471 plus 5 μmol/kg of INCB3284 (grey diamonds, n=6). Arrow indicates vehicle/drug administration and start of the resuscitation period. HEM: hemorrhage period. Data are mean±SE (FIGS. 6A-6H) or survival percentages (FIG. 6I). *, % and #denote p<0.05 vs. vehicle treated animals, as indicated in the graphs. FIG. 6A. Blood volume hemorrhaged as percentage of total blood volume (% TBV). FIG. 6B. Systolic blood pressure (SBP, mmHg). FIG. 6C. Diastolic blood pressure (DBP, mmHg). FIG. 6D. Mean arterial blood pressure (MAP, mmHg). FIG. 6E. Lactate concentrations (mmol/L). FIG. 6F. Cumulative resuscitation fluid requirements (mL/kg). FIG. 6G. Hematocrit (%). FIG. 6H. PaO₂, partial pressure of oxygen in arterial blood (mmHg). FIG. 6I. Survival (%).

FIGS. 7A-7I provide graphs demonstrating the effects of BX471 and INCB3284 on serum markers of inflammation. Serum markers of inflammation were measured at t=0 min (baseline) and following hemorrhage and resuscitation (H/R) in animals surviving greater than 5 h. Open bars: vehicle treated animals, n=5. H/R samples were obtained at t=390 min (n=3), t=350 min (n=1) and t=330 min (n=1). Light grey bars: BX471 treated animals, n=5. All H/R samples were obtained at t=390 min. Dark grey bars: INCB3284 treated animals, n=4. H/R samples were obtained at t=390 min (n=3) and t=360 min (n=1). Inflammation marker concentrations are expressed as OD per mg of total protein (OD/mg). Data are mean±SE. *: p<0.05 for baseline vs. H/R. #: p<0.05 for H/R vehicle vs. H/R BX471 or H/R INCB3284. FIG. 7A. TNFα. FIG. 7B. IL6. FIG. 7C. IFNγ. FIG. 7D. IL1α. FIG. 7E. IL1β. FIG. 7F. CCL2. FIG. 7G. CCL3. FIG. 7H. Combined inflammation markers that showed significant increases from baseline to H/R (FIGS. 7A-7G). FIG. 7I. CCL5.

FIGS. 8A-8D provide graphs demonstrating the effects of BX471 and INCB3284 on shock tolerance. Animals were hemorrhaged to MAP of 30 mmHg for 45 min followed by an observation period of 180 min. At t=45 min, animals received either vehicle (n=8, open circles/bar), 0.5 μmol/kg BX471 (n=7, light grey squares/bar) or 5 μmol/kg INCB3284 (n=5, dark grey triangles/bar) without further intervention. Arrows indicate time of vehicle/drug administration. HEM: hemorrhage period. Data are Data are mean±SE (FIGS. 8A-8C) or percentage survival (FIG. 8D). FIG. 8A. Blood volume hemorrhaged as percentage of total blood volume (% TBV). FIG. 8B. Lactate concentrations at t=O and t=45 min. FIG. 8C. Mean arterial blood pressure (mmHg). FIG. 8D. Survival (%).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular examples only and is not intended to be limiting.

### Definitions

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

It should be noted that amounts and other numerical data can be expressed herein in a range format. It can be further understood that the endpoints of each of the ranges are significant both in relation to the other endpoint and independent of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y’, and ‘about z’ as well as the ranges of ‘less than x,’ less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y′, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Examples defined by each of these transition terms are within the scope of this disclosure.

A “control” is an alternative subject or sample used in an experiment for comparison purposes. A control can be “positive” or “negative.”

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Optional” or “optionally” means that the subsequently described event or circumstance may or may not occur, and that the description includes instances where said event or circumstance occurs and instances where it does not.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., lactate concentration). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

A “pharmaceutically acceptable” component can refer to a component that is not biologically or otherwise undesirable, e.g., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising a therapeutic agent) refers to an amount that is effective to achieve a desired therapeutic result. In some examples, a desired therapeutic result is the restoration of hemodynamic parameters of stable circulation following hemorrhagic shock. In some examples, a desired therapeutic result is the reduction of inflammatory markers such as TNFα and/or IL-6 following hemorrhagic shock. In some examples, a desired therapeutic result is the reduction of fluid requirements following hemorrhagic shock. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Compositions

### Chemokine Receptors (CCR)

Chemokines and their receptors are known to play a useful role in inflammation, regulating functions including cell migration, cell proliferation, tissue repair, and angiogenesis (Pozzi S, Satchi-Fainaro R. The role of CCL2/CCR2 axis in cancer and inflammation: The next frontier in nanomedicine. Adv Drug Deliv Rev. 2024; 209:115318. doi: 10.1016/j.addr.2024.115318. PubMed PMID: 38643840). Numerous chemokine receptor antagonists have been developed and explored as potential therapeutics for inflammatory disease processes (Lai, W. Y.; Mueller, A. Latest update on chemokine receptors as therapeutic targets. Biochem. Soc. Trans. 2021, 49, 1385-1395).

The CCL2/CCR2 axis has recently been identified as a potential therapeutic target in both acute and chronic processes such as ischemia-reperfusion injury, cardiovascular disease, and cancer associated inflammation (Id.; Geng H, Chen L, Tang J, Chen Y, Wang L. The Role of CCL2/CCR2 Axis in Cerebral Ischemia-Reperfusion Injury and Treatment: From Animal Experiments to Clinical Trials. Int J Mol Sci. 2022; 23(7). doi: 10.3390/ijms23073485. PubMed PMID: 35408846; PubMed Central PMCID: PMC8998625). Furthermore, the effects of CCR2, CCR3, and CCR5 antagonists were tested in rat models of hemorrhagic shock and subsequent fluid resuscitation (DeSantis, A. J.; Weche, M.; Enten, G. A.; Gao, X.; Majetschak, M. The Chemokine (C-C Motif) Receptor 2 Antagonist INCB3284 Reduces Fluid Requirements and Protects From Hemodynamic Decompensation During Resuscitation from Hemorrhagic Shock. Crit. Care Explor. 2022, 4, e0701; Weche, M.; DeSantis, A. J.; McGee, M. Y.; Enten, G. A.; Gao, X.; Majetschak, M. Effects of chemokine (C-C motif) receptor 2 and 3 antagonists in rat models of hemorrhagic shock. PLoS ONE 2023, 18, e0284472). While the CCR5 antagonist Maraviroc did not affect fluid resuscitation requirements, fluid-sparing effects of the CCR2 antagonist INCB3284 and the CCR3 antagonist SB328437 were observed. Although the fluid-sparing effects of SB328437 were observed in short resuscitation periods after hemorrhagic shock, INCB3284 showed beneficial effects during resuscitation for up to 4 h after hemorrhagic shock, and dosing optimization improved its therapeutic efficacy (Id.). These observations suggest that activation of CCR2 and CCR3 contribute to the development of increased resuscitation fluid requirements after hemorrhagic shock.

Chemokines and their receptors are well known to be promiscuous in their receptor and ligand specificity, respectively (Alexander, S. P. H.; Christopoulos, A.; Davenport, A. P.; Kelly, E.; Mathie, A. A.; Peters, J. A.; Veale, E. L.; Armstrong, J. F.; Faccenda, E.; Harding, S. D.; et al. The Concise Guide to Pharmacology 2023/24: G protein-coupled receptors. Br. J. Pharmacol. 2023, 180 (Suppl. S2), S23-S144; Zlotnik, A.; Yoshie, O.; Uddin, M.; Thelen, M. Chemokine receptors (version 2020.5) in the IUPHAR/BPS Guide to Pharmacology Database. In IUPHAR/BPS Guide to Pharmacology CITE; University of Edinburgh: Edinburgh, UK, 2020; Alexander, S. P.; Christopoulos, A.; Davenport, A. P.; Kelly, E. The Concise Guide to Pharmacology 2017/18: G protein-coupled receptors. Br. J. Pharmacol. 2017, 174 (Suppl. S1), S17-S129; Saha, S.; Sano, F.K.; Sharma, S.; Ganguly, M.; Mishra, S.; Dalal, A.; Akasaka, H.; Kobayashi, T.A.; Zaidi, N.; Tiwari, D.; et al. Molecular basis of promiscuous chemokine binding and structural mimicry at the C-X-C chemokine receptor, CXCR2. Mol. Cell 2025, 85, 976-988.e9). With numerous shared ligands between chemokine receptors creating redundancy in the system, the efficacy and translation to clinical practice of monotherapies targeting specific chemokines and/or their receptors have been challenging (Noels H, Weber C, Koenen R R. Chemokines as Therapeutic Targets in Cardiovascular Disease. Arterioscler Thromb Vasc Biol. 2019; 39(4):583-92. doi: 10.1161/ATVBAHA.118.312037. PubMed PMID: 30760014).

### CCR1

Chemokine (C-C motif) receptor 1 (CCR1) is expressed in numerous cell types and tissues, including leukocytes, endothelial cells and vascular smooth muscle cells, and is thought to play useful roles in the pathogenesis of multiple disease processes, including rheumatoid arthritis, multiple sclerosis, eosinophilic airway inflammation, and multiple myeloma (Alexander, S. P. H.; Christopoulos, A.; Davenport, A. P.; Kelly, E.; Mathie, A. A.; Peters, J. A.; Veale, E. L.; Armstrong, J. F.; Faccenda, E.; Harding, S. D.; et al. The Concise Guide to Pharmacology 2023/24: G protein-coupled receptors. Br. J. Pharmacol. 2023, 180 (Suppl. S2), S23-S144; Hwang, J.; Son, K. N.; Kim, C. W.; Ko, J.; Na, D. S.; Kwon, B. S.; Gho, Y. S.; Kim, J. Human CC chemokine CCL23, a ligand for CCR1, induces endothelial cell migration and promotes angiogenesis. Cytokine 2005, 30, 254-263; Hayes, I. M.; Jordan, N. J.; Towers, S.; Smith, G.; Paterson, J. R.; Earnshaw, J. J.; Roach, A. G.; Westwick, J.; Williams, R. J. Human vascular smooth muscle cells express receptors for CC chemokines. Arterioscler. Thromb. Vasc. Biol. 1998, 18, 397-403; Koelink, P. J.; Overbeek, S. A.; Braber, S.; de Kruijf, P.; Folkerts, G.; Smit, M. J.; Kraneveld, A. D. Targeting chemokine receptors in chronic inflammatory diseases: An extensive review. Pharmacol. Ther. 2012, 133, 1-18; Du, X.; Li, F.; Zhang, C.; Li, N.; Huang, H.; Shao, Z.; Zhang, M.; Zhan, X.; He, Y.; Ju, Z.; et al. Eosinophil-derived chemokine (hCCL15/23, mCCL6) interacts with CCR1 to promote eosinophilic airway inflammation. Signal Transduct. Target. Ther. 2021, 6, 91; Gilchrist, A.; Echeverria, S.L. Targeting Chemokine Receptor CCR1 as a Potential Therapeutic Approach for Multiple Myeloma. Front. Endocrinol. 2022, 13, 846310). CCR1 is evolutionary closely related to CCR2 and CCR3 (Nomiyama, H.; Osada, N.; Yoshie, O. A family tree of vertebrate chemokine receptors for a unified nomenclature. Dev. Comp. Immunol. 2011, 35, 705-715; Dyer, D.P.; Medina-Ruiz, L.; Bartolini, R.; Schuette, F.; Hughes, C. E.; Pallas, K.; Vidler, F.; Macleod, M. K. L.; Kelly, C. J.; Lee, K. M.; et al. Chemokine Receptor Redundancy and Specificity Are Context Dependent. Immunity 2019, 50, 378-389.e5), and shares multiple chemokine ligands, such as chemokine (C-C motif) ligand 5 (CCL5), CCL7, CCL8, CCL13, or CCL16, with CCR2 and CCR3 in humans and rodents (Id.).

Previous findings indicate that systemic concentrations of CCL3, an agonist at CCR1 and CCR5, CCL5, an agonist at CCR1 and CCR3, as well as CCL7, which activates CCR1, CCR2, CCR3, and CCR5, increase in vehicle-treated animals under the experimental conditions (Id.). This can suggest that the release of endogenous CCR1 agonists during hemorrhagic shock and resuscitation contributes to increased systemic fluid requirements. As such, it appears possible that the activation of CCR1 can also affect resuscitation fluid requirements after hemorrhage, and that the blockade of CCR1 can possess therapeutic potential in pre-clinical models of hemorrhagic shock.

Disclosed herein is a resuscitation fluid comprising a CCR1 inhibitor and an aqueous iso or hypertonic salt solution. In some examples, the CCR1 inhibitor of the compositions and methods disclosed herein can be BX471 (ZK-811752 or (R)-1-(5-chloro-2-(2-(4-(4-fluorobenzyl)-2-methylpiperazin-1-yl)-2-oxoethoxy)phenyl)urea), CCX354 (2-(3-(1H-imidazol-2-yl)-1H-pyrazolo[3,4-b]pyridin-1-yl)-1-(4-(4-chloro-3-methoxyphenyl)piperazin-1-yl)ethan-1-one), BMS-817399 (1-((R)-1-((S)-4-(4-chlorophenyl)-4-hydroxy-3,3-dimethylpiperidin-1-yl)-3-methyl-1-oxobutan-2-yl)-3-(2-hydroxy-2-methylpropyl)urea), MLN3897 ((4S)-4-(4-Chlorophenyl)-1-((3E)-3-(9-(1-hydroxy-1-methyl-ethyl)-5H-(1)benzoxepino(3,4-b)pyridin-1-ylidene)propyl)-3,3-dimethyl-piperidin-4-ol), CP-481715 (N-((2S,3S,5R)-5-carbamoyl-1-(3-fluorophenyl)-3,8-dihydroxy-8-methylnonan-2-yl)quinoxaline-2-carboxamide), pharmaceutically acceptable salts thereof, or any combination thereof.

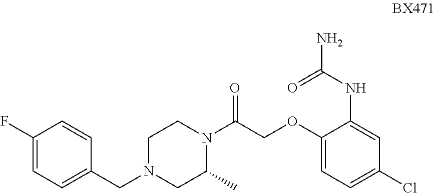 [SMILES: C[C@@H]1CN(Cc2ccc(F)cc2)CCN1C(=O)COc1ccc(Cl)cc1NC(N)=O]

Potent CCR1 antagonists have been developed, and several have entered clinical trials, including BX471, which was tested for therapeutic potential in patients with multiple sclerosis (Gladue, R. P.; Brown, M. F.; Zwillich, S. H. CCR1 antagonists: What have we learned from clinical trials. Curr. Top. Med. Chem. 2010, 10, 1268-1277; Zipp, F.; Hartung, H. P.; Hillert, J.; Schimrigk, S.; Trebst, C.; Stangel, M.; Infante-Duarte, C.; Jakobs, P.; Wolf, C.; Sandbrink, R.; et al. Blockade of chemokine signaling in patients with multiple sclerosis. Neurology 2006, 67, 1880-1883). BX471 is an orally active non-peptide CCR1 antagonist with high selectivity (Liang, M.; Mallari, C.; Rosser, M.; Ng, H.P.; May, K.; Monahan, S.; Bauman, J.G.; Islam, I.; Ghannam, A.; Buckman, B.; et al. Identification and characterization of a potent, selective, and orally active antagonist of the CC chemokine receptor-1. J. Biol. Chem. 2000, 275, 19000-19008), and beneficial effects of BX471 have been reported in several preclinical models, such as pancreatitis, sepsis or allograft rejection models (Horuk, R.; Clayberger, C.; Krensky, A.M.; Wang, Z.; Grone, H.-J.; Weber, C.; Weber, K. S. C.; Nelson, P. J.; May, K.; Rosser, M.; et al. A non-peptide functional antagonist of the CCR1 chemokine receptor is effective in rat heart transplant rejection. J. Biol. Chem. 2001, 276, 4199-4204; He, M.; Horuk, R.; Bhatia, M. Treatment with BX471, a nonpeptide CCR1 antagonist, protects mice against acute pancreatitis-associated lung injury by modulating neutrophil recruitment. Pancreas 2007, 34, 233-241; He, M.; Horuk, R.; Moochhala, S. M.; Bhatia, M. Treatment with BX471, a CC chemokine receptor 1 antagonist, attenuates systemic inflammatory response during sepsis. Am. J. Physiol. Gastrointest. Liver Physiol. 2007, 292, G1173-G1180). A 16-week dosing of BX471 was found to be well tolerated (Id.), and BX471 demonstrates a safety and toxicity profile that permits its use in humans (Elices, M. J. BX-471 Berlex. Curr. Opin. Investig. Drugs 2002, 3, 865-869).

Observations that hematocrit values were comparable among groups and that lactate concentrations declined to a similar degree in vehicle and BX471-treated animals indicate that all animals were resuscitated to a comparable intravascular volume status. While BX471-treatment significantly reduced the wet-to-dry (W/D) ratios of the large intestine, the W/D ratios of the small intestine and the lung were lower with BX471 treatment, but these differences did not reach a level of statistical significance. Because the W/D ratios of the sum of all tissues that were evaluated were also significantly reduced with BX471 treatment, the observations point toward the systemic reduction in the third-spacing of fluids as a possible mechanism through which BX471 reduced fluid requirements, rather than selective effects of BX471 in the large intestine.

Disclosed herein is a resuscitation fluid comprising BX471 or a pharmaceutically acceptable salt thereof and an aqueous iso or hypertonic salt solution.

### CCR1 Combinations

The fluid-sparing effects of BX471 mimicked the previous observations on the effects of the CCR2 antagonist INCB3284 and the CCR3 antagonist SB328437 in the same animal model (Id.). It was shown that the iv administration of BX471 at the beginning of fluid resuscitation from hemorrhagic shock significantly reduces the fluid requirements to stabilize hemodynamics in rat models. These fluid-sparing effects occurred dose-dependently and were not associated with noticeable adverse events. This suggests that the activation of CCR1, CCR2, and CCR3 result in similar pathophysiological consequences after hemorrhagic shock and fluid resuscitation and can imply at least some degree of redundancy regarding their effects on cardiovascular function.

Disclosed herein is a resuscitation fluid comprising a CCR1 inhibitor, an aqueous iso or hypertonic salt solution, and a CCR inhibitor, such as, for example, a CCR2 inhibitor, a CCR3 inhibitor, a CCR5 inhibitor, or combinations thereof. Disclosed herein is a resuscitation fluid comprising a CCR1 inhibitor, an aqueous iso or hypertonic salt solution, and a CCR2 inhibitor. Disclosed herein is a resuscitation fluid comprising BX471 or a pharmaceutically acceptable salt thereof, an aqueous iso or hypertonic salt solution, and a CCR inhibitor, such as, for example, a CCR2 inhibitor, a CCR3 inhibitor, a CCR5 inhibitor, or combinations thereof. Disclosed herein is a resuscitation fluid comprising BX471 or a pharmaceutically acceptable salt thereof, an aqueous iso or hypertonic salt solution, and a CCR2 inhibitor.

### CCR1+CCR3

Disclosed herein is a resuscitation fluid comprising a CCR1 inhibitor, an aqueous iso or hypertonic salt solution, and a CCR3 inhibitor. Disclosed herein is a resuscitation fluid comprising BX471 or a pharmaceutically acceptable salt thereof, an aqueous iso or hypertonic salt solution, and a CCR3 inhibitor. In some examples, the CCR3 inhibitor of the compositions and methods disclosed herein can be AKST4290 (ALK4290 or (R)-2-(1-(1-(4-chloro-3-methylbenzyl)piperidin-4-yl)-5-oxopyrrolidine-2-carboxamido)-N,N,6-trimethylisonicotinamide), SB328437 (methyl (S)-2-(1-naphthamido)-3-(4-nitrophenyl)propanoate), pharmaceutically acceptable salts, or combinations thereof. In some examples, the CCR3 inhibitor disclosed herein can be SB328437 or a pharmaceutically acceptable salt thereof.

### CCR1+CCR5

Disclosed herein is a resuscitation fluid comprising a CCR1 inhibitor, an aqueous iso or hypertonic salt solution, and a CCR5 inhibitor. Disclosed herein is a resuscitation fluid comprising BX471 or a pharmaceutically acceptable salt thereof, an aqueous iso or hypertonic salt solution, and a CCR5 inhibitor. In some examples, the CCR5 inhibitor of the compositions and methods disclosed herein can be maraviroc (4,4-Difluoro-N-[(1S)-3-{(1R,3s,5S)-3-[3-methyl-5-(propan-2-yl)-4H-1,2,4-triazol-4-yl]-8-azabicyclo[3.2.1]octan-8-yl}-1-phenylpropyl]cyclohexanecarboxamide), TAK-779 (N,N-dimethyl-N-(4-(2-(p-tolyl)-6,7-dihydro-5H-benzo[7]annulene-8-carboxamido)benzyl)tetrahydro-2H-pyran-4-aminium chloride), cenicriviroc ((S,E)-8-(4-(2-butoxyethoxy)phenyl)-1-isobutyl-N-(4-(((1-propyl-1H-imidazol-5-yl)methyl)sulfinyl)phenyl)-1,2,3,4-tetrahydrobenzo[b]azocine-5-carboxamide), LUF8100, BMS-813160 (N-((1R,2S,5R)-2-((S)-3-((7-(tert-butyl)pyrazolo[1,5-a][1,3,5]triazin-4-yl)amino)-2-oxopyrrolidin-1-yl)-5-(tert-butylamino)cyclohexyl)acetamide), TAK-220 (1-acetyl-N-(3-(4-(4-carbamoylbenzyl)piperidin-1-yl)propyl)-N-(3-chloro-4-methylphenyl)piperidine-4-carboxamide), pharmaceutically acceptable salts, or combinations thereof.

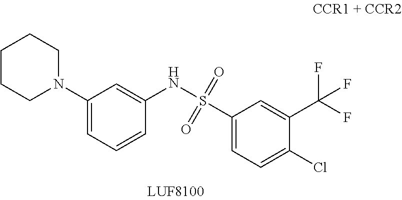 [SMILES: O=S(=O)(Nc1cccc(N2CCCCC2)c1)c1ccc(Cl)c(C(F)(F)F)c1]

Furthermore, CCR1 and CCR2 were identified as drug targets to improve fluid resuscitation after hemorrhagic shock (Id.). The selective CCR1 antagonist BX471 as well as the selective CCR2 antagonist INCB3284 were shown to significantly reduce fluid requirements to maintain hemodynamics in rat models of hemorrhagic shock (Id.). The effects of BX471 and INCB3284 after hemorrhagic shock, however, have not been directly compared with each other, and the consequences of simultaneous blockade of CCR1 and CCR2 during fluid resuscitation after hemorrhage were unknown. In addition, potential effects of BX471 on shock tolerance have not previously been evaluated. Rat models were used to compare fluid sparing effects of BX471 and INCB3284 during resuscitation after hemorrhagic shock, to evaluate the effects of co-administration of BX471 and INCB3284, and to test whether each chemokine receptor antagonist modulates shock tolerance after hemorrhage in the absence of fluid resuscitation. The observations that hematocrit values, lactate concentrations and PaO₂ were indistinguishable among the groups suggest that right heart failure due to acute fluid overload or hypoxemia are unlike to account for mortality, which point toward loss of vascular tone with subsequent circulatory failure as a reason for mortality, a common event during resuscitation from hemorrhagic shock (Gomez H, Mesquida J, Hermus L, Polanco P, Kim H K, Zenker S, et al. Physiologic responses to severe hemorrhagic shock and the genesis of cardiovascular collapse: can irreversibility be anticipated? The Journal of surgical research. 2012; 178(1):358-69. Epub 2012/04/06. doi: 10.1016/j.jss.2011.12.015. PubMed PMID: 22475354; PubMed Central PMCID: PMC3424406). In contrast to the fluid sparing effects of BX471 and INCB3287 in the resuscitation model, both chemokine receptor antagonists did not affect survival times after hemorrhage in the absence of fluid resuscitation. These observations were consistent with previous findings on the effects of INCB3284 in a model that mimicked continuous hemorrhage without fluid resuscitation (Id.). Both drugs were found to reduce fluid requirements while hematocrit values were indistinguishable from vehicle-treated animals, which can suggest reduction of third-spacing of fluids and can point toward modulation of endothelial function as one potential mechanism. Such a mechanism would be consistent with previous findings suggesting that CCR1 and CCR2 contribute to the development of vascular leakiness and edema formation in disease processes (Roblek M, Protsyuk D, Becker P F, Stefanescu C, Gorzelanny C, Glaus Garzon J F, et al. CCL2 Is a Vascular Permeability Factor Inducing CCR2-Dependent Endothelial Retraction during Lung Metastasis. Mol Cancer Res. 2019; 17(3):783-93. Epub 2018/12/16. doi: 10.1158/1541-7786.MCR-18-0530. PubMed PMID: 30552233; PubMed Central PMCID: PMC6445360; Yan J, Xu W, Lenahan C, Huang L, Ocak U, Wen J, et al. Met-RANTES preserves the blood-brain barrier through inhibiting CCR1/SRC/Rac1 pathway after intracerebral hemorrhage in mice. Fluids Barriers CNS. 2022; 19(1):7. Epub 20220121. doi: 10.1186/s12987-022-00305-3. PubMed PMID: 35062973; PubMed Central PMCID: PMC8781527; Yang D, Tong L, Wang D, Wang Y, Wang X, Bai C. Roles of CC chemokine receptors (CCRs) on lipopolysaccharide-induced acute lung injury. Respir Physiol Neurobiol. 2010; 170(3):253-9. Epub 20100210. doi: 10.1016/j.resp.2010.02.002. PubMed PMID: 20152938). Alternatively, both chemokine receptors have been described to modulate vascular smooth muscle responsiveness to stress hormones, which could also have contributed to the observed effect (Id.; Gao X, DeSantis A J, Enten G A, Weche M, Marcet J E, Majetschak M. Heteromerization between alpha(1B)—adrenoceptor and chemokine (C-C motif) receptor 2 biases alpha(1B)-adrenoceptor signaling: Implications for vascular function. FEBS Lett. 2022; 596(20):2706-16. Epub 2022/08/04. doi: 10.1002/1873-3468.14463. PubMed PMID: 35920096; PubMed Central PMCID: PMCPMC9830583; Gao X, Majetschak M. Molecular Boolean analyses of chemokine (C-C motif) receptor 1, alpha(1B)-adrenoceptor and arginine vasopressin receptor 1A heteromers. Biochem Biophys Rep. 2026; 45:102404. Epub 20251205. doi: 10.1016/j.bbrep.2025.102404. PubMed PMID: 41867271; PubMed Central PMCID: PMCPMC13005234). Because CCR1 and CCR2 are closely related chemokine receptors that share multiple endogenous ligands, similar mechanisms underlying their fluid sparing effects could appear possible.

Disclosed herein is a resuscitation fluid comprising a CCR1 inhibitor, an aqueous iso or hypertonic salt solution, and a CCR2 inhibitor. Disclosed herein is a resuscitation fluid comprising BX471 or a pharmaceutically acceptable salt thereof, an aqueous iso or hypertonic salt solution, and a CCR2 inhibitor. In some examples, the CCR2 inhibitor of the compositions and methods disclosed herein can be RS 504393 (6-methyl-1′-(2-(5-methyl-2-phenyloxazol-4-yl)ethyl)spiro[benzo[d][1,3]oxazine-4,4′-piperidin]-2(1H)-one), INCB3344 (N-(2-(((3S,4S)-1-((1r,4S)-4-(benzo[d][1,3]dioxol-5-yl)-4-hydroxycyclohexyl)-4-ethoxypyrrolidin-3-yl)amino)-2-oxoethyl)-3-(trifluoromethyl)benzamide), TAK-779 (N,N-dimethyl-N-(4-(2-(p-tolyl)-6,7-dihydro-5H-benzo[7]annulene-8-carboxamido)benzyl)tetrahydro-2H-pyran-4-aminium chloride), abaucin (1′-(4-(trifluoromethyl)phenethyl)spiro[benzo[d][1,3]oxazine-4,4′-piperidin]-2(1H)-one), INCB3284 (N-(2-(((R)-1-((1s,4S)-4-hydroxy-4-(6-methoxypyridin-3-yl)cyclohexyl)pyrrolidin-3-yl)amino)-2-oxoethyl)-3-(trifluoromethyl)benzamide), pharmaceutically acceptable salts, or combinations thereof. In some examples, the CCR2 inhibitor can be INCB3284 or a pharmaceutically acceptable salt thereof.

### Iso or Hypertonic Salt Solutions

In some examples, the iso or hypertonic salt solution of the compositions and methods disclosed herein can be Ringer's solution or lactated Ringer's (LS) solution, although normal saline or other similar isotonic crystalloid solutions can be used. For example, isotonic fluid replacement solutions can include solutions where isotonic crystalloid solutions are mixed with macromolecular solutions of plasma proteins or synthesized molecules with similar oncotic properties (colloids) including albumin, dextran, hetastarch or polygelatin in NaCl. In some examples, the isotonic crystalloid fluid disclosed herein can be, for example, 0.9% sodium chloride or normal saline, 0.45% sodium chloride or half normal saline, 3% sodium chloride or hypertonic saline, Ringer's solution, lactated Ringer's solution, dextrose 5% in water (D5W), or acetate-based infusion fluids such as PlasmaLyte™, Hartmann's™, or acetated Ringer's solution.

In some examples, the ionic salt of the present fluid of the compositions and methods disclosed herein can be a pharmaceutically acceptable salt that ionizes to provide osmotic pressure in an aqueous solution. For example, salts can include sodium chloride (NaCl), potassium chloride (KCl), or combinations thereof. In some examples, NaCl can be used as the agent to augment intravascular fluid. In some examples, the salt is in the form of a saline solution. Those having ordinary skill in the art will recognize that saline is a solution of NaCl in sterile water, used commonly for intravenous infusion.

### Formulations

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

The dose administered to a patient, particularly a human, should be sufficient to achieve a therapeutic response in the patient over a reasonable time frame, without lethal toxicity, and causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The disclosed resuscitation fluid can also be used as a storage solution for organs during organ transplant, for wound irrigation, as a solution for urological and gynecological procedures, to treat intracranial hypertension from head injury, and to help reduce contrast induced nephrotoxicity. Pre-hospital uses for the present resuscitation fluid include ambulance use, battlefield use, emergency room use, trauma, and intensive care use. Similarly, the present resuscitation fluid can be utilized for veterinary use in the same manner it is utilized for human use.

### Methods

### Patient

In some examples, disclosed herein are methods of fluid resuscitation of a patient in need thereof comprising administering to the patient a resuscitation fluid and a therapeutically effective amount of a CCR1 inhibitor. In some examples, the methods disclosed herein further comprise administering a therapeutically effective amount of an additional CCR inhibitor, such as a CCR2 inhibitor, a CCR3 inhibitor, a CCR5 inhibitor, or combinations thereof. In some examples, disclosed herein are methods of fluid resuscitation of a patient in need thereof comprising administering to the patient a resuscitation fluid, a therapeutically effective amount of a CCR1 inhibitor, and a therapeutically effective amount of a CCR2 inhibitor. In some examples, disclosed herein are methods of fluid resuscitation of a patient in need thereof comprising administering to the patient a resuscitation fluid, a therapeutically effective amount of BX471 or a pharmaceutically acceptable salt thereof, and a therapeutically effective amount of a CCR2 inhibitor. In some examples, disclosed herein are methods of fluid resuscitation of a patient in need thereof comprising administering to the patient a resuscitation fluid and a therapeutically effective amount of BX471 or a pharmaceutically acceptable salt thereof. In some examples, disclosed herein are methods of fluid resuscitation of a patient in need thereof comprising administering to the patient a resuscitation fluid and a therapeutically effective amount of BX471 or a pharmaceutically acceptable salt thereof, and a therapeutically effective amount of INCB3284 or a pharmaceutically acceptable salt thereof.

In some examples, disclosed are methods of reducing the resuscitation fluid requirements for fluid resuscitation by administering to a patient in need of fluid resuscitation a resuscitation fluid and a therapeutically effective amount of a CCR1 inhibitor. In some examples, the methods disclosed herein further comprise administering a therapeutically effective amount of an additional CCR inhibitor, such as a CCR2 inhibitor, a CCR3 inhibitor, a CCR5 inhibitor, or combinations thereof. Also disclosed are methods of reducing the resuscitation fluid requirements for fluid resuscitation by administering to a patient in need of fluid resuscitation a resuscitation fluid, a therapeutically effective amount of a CCR1 inhibitor, and a therapeutically effective amount of a CCR2 inhibitor. In some examples, disclosed are methods of reducing the resuscitation fluid requirements for fluid resuscitation by administering to a patient in need of fluid resuscitation a resuscitation fluid, a therapeutically effective amount of BX471 or a pharmaceutically acceptable salt thereof, and a therapeutically effective amount of a CCR2 inhibitor. In some examples, disclosed are methods of reducing the resuscitation fluid requirements for fluid resuscitation by administering to a patient in need of fluid resuscitation a resuscitation fluid and a therapeutically effective amount of BX471 or a pharmaceutically acceptable salt thereof. In some examples, disclosed are methods of reducing the resuscitation fluid requirements for fluid resuscitation by administering to a patient in need of fluid resuscitation a resuscitation fluid, a therapeutically effective amount of BX471 or a pharmaceutically acceptable salt thereof, and a therapeutically effective amount of INCB3284 or a pharmaceutically acceptable salt thereof.

Also disclosed are methods of preventing or delaying hemodynamic decompensation in patients with shock comprising administering to a patient in need thereof a resuscitation fluid and a therapeutically effective amount of a CCR1 inhibitor. In some examples, the methods disclosed herein further comprise administering a therapeutically effective amount of an additional CCR inhibitor, such as a CCR2 inhibitor, a CCR3 inhibitor, a CCR5 inhibitor, or combinations thereof. Disclosed are methods of preventing or delaying hemodynamic decompensation in patients with shock comprising administering to a patient in need thereof a resuscitation fluid, a therapeutically effective amount of a CCR1 inhibitor, and a therapeutically effective amount of a CCR2 inhibitor. In some examples, disclosed are methods of preventing or delaying hemodynamic decompensation in patients with shock comprising administering to a patient in need thereof a resuscitation fluid, a therapeutically effective amount of BX471 or a pharmaceutically acceptable salt thereof, and a therapeutically effective amount of a CCR2 inhibitor. In some examples, disclosed are methods of preventing or delaying hemodynamic decompensation in patients with shock comprising administering to a patient in need thereof a resuscitation fluid and a therapeutically effective amount of BX471 or a pharmaceutically acceptable salt thereof. In some examples, disclosed are methods of preventing or delaying hemodynamic decompensation in patients with shock comprising administering to a patient in need thereof a resuscitation fluid, a therapeutically effective amount of BX471 or a pharmaceutically acceptable salt thereof, and a therapeutically effective amount of INCB3284 or a pharmaceutically acceptable salt thereof.

In some examples, disclosed are methods for preventing or treating septic shock, hemorrhagic shock, hypotension, acidosis, and/or hypovolemia comprising administering to a patient in need of treatment or prevention thereof a resuscitation fluid and a therapeutically effective amount of a CCR1 inhibitor. In some examples, the methods disclosed herein further comprise administering a therapeutically effective amount of an additional CCR inhibitor, such as a CCR2 inhibitor, a CCR3 inhibitor, a CCR5 inhibitor, or combinations thereof. In some examples, disclosed are methods for preventing or treating septic shock, hemorrhagic shock, hypotension, acidosis, and/or hypovolemia comprising administering to a patient in need of treatment or prevention thereof a resuscitation fluid, a therapeutically effective amount of a CCR1 inhibitor, and a therapeutically effective amount of a CCR2 inhibitor. In some examples, disclosed are methods for preventing or treating septic shock, hemorrhagic shock, hypotension, acidosis, and/or hypovolemia comprising administering to a patient in need of treatment or prevention thereof a resuscitation fluid, a therapeutically effective amount of BX471 or a pharmaceutically acceptable salt thereof, and a therapeutically effective amount of a CCR2 inhibitor. In some examples, disclosed are methods for preventing or treating septic shock, hemorrhagic shock, hypotension, acidosis, and/or hypovolemia comprising administering to a patient in need of treatment or prevention thereof a resuscitation fluid and BX471 or a pharmaceutically acceptable salt thereof. In some examples, disclosed are methods for preventing or treating septic shock, hemorrhagic shock, hypotension, acidosis, and/or hypovolemia comprising administering to a patient in need of treatment or prevention thereof a resuscitation fluid, BX471 or a pharmaceutically acceptable salt thereof, and INCB3284 or a pharmaceutically acceptable salt thereof.

In some examples, disclosed are methods for preventing or treating hemorrhagic shock, comprising administering to a patient in need of treatment or prevention thereof a resuscitation fluid and a therapeutically effective amount of a CCR1 inhibitor. In some examples, the methods disclosed herein further comprise administering a therapeutically effective amount of an additional CCR inhibitor, such as a CCR2 inhibitor, a CCR3 inhibitor, a CCR5 inhibitor, or combinations thereof. In some examples, disclosed are methods for preventing or treating hemorrhagic shock, comprising administering to a patient in need of treatment or prevention thereof a resuscitation fluid, a therapeutically effective amount of a CCR1 inhibitor, and a therapeutically effective amount of a CCR2 inhibitor. In some examples, disclosed are methods for preventing or treating hemorrhagic shock, comprising administering to a patient in need of treatment or prevention thereof a resuscitation fluid, a therapeutically effective amount of BX471 or a pharmaceutically acceptable salt thereof, and a therapeutically effective amount of a CCR2 inhibitor. In some examples, disclosed are methods for preventing or treating hemorrhagic shock comprising administering to a patient in need of treatment or prevention thereof a resuscitation fluid and BX471 or a pharmaceutically acceptable salt thereof. In some examples, disclosed are methods for preventing or treating hemorrhagic shock, comprising administering to a patient in need of treatment or prevention thereof a resuscitation fluid, a therapeutically effective amount of BX471 or a pharmaceutically acceptable salt thereof, and a therapeutically effective amount of INCB3284 or a pharmaceutically acceptable salt thereof.

In some examples, suitable patients that can be treated with the disclosed methods can be patients having hemorrhagic shock, e.g., traumatic-hemorrhagic shock. In some examples, suitable patients can be those undergoing cardiovascular, abdominal, or transplant surgery, e.g., a percutaneous coronary intervention, a cardiac bypass surgery, a fibrinolytic therapy, or a stent placement. Patients being treated for ischemia, hypovolemic shock, myocardial infarction, or stroke are also suitable patients for the disclosed methods. In further examples, the patients can be treated for trauma, burn, sepsis, or shock. In some examples, suitable patients can be those with complications of fluid overload, such as patients with acute kidney injury, pulmonary edema, cardiac failure, delayed wound healing, tissue breakdown, and impaired bowel function. In some examples, suitable patients can be hypotensive and/or hemodynamically instable patients.

### Reduction of Inflammatory Markers

While serum concentrations of CCL5 increased in BX471-treated animals to a degree observed in vehicle-treated animals, significant increases in CCL3 and CCL7 concentrations could not be detected after BX471 treatment. This finding could be explained with a feed-forward mechanism, by which CCL5, via activation of CCR1, amplifies the acute inflammatory response and induces the systemic release of CCL3 and CCL7. This assumption would be consistent with the previously described effects of CCR1 knockout on the expression of CCL3 after renal ischemia-reperfusion injury (Furuichi, K.; Gao, J. L.; Horuk, R.; Wada, T.; Kaneko, S.; Murphy, P. M. Chemokine receptor CCR1 regulates inflammatory cell infiltration after renal ischemia-reperfusion injury. J. Immunol. 2008, 181, 8670-8676). The finding that CCL5 concentrations were not affected by BX471 treatment is in agreement with previous findings, which showed that CCL5 concentrations are already significantly increased at time points before BX471 was administered (Id.). The findings that there were no differences in the concentrations of a panel of inflammation markers in the small intestine and lung extracts from vehicle-treated and BX471-treated animals matched observations regarding the W/D ratios in these tissues. In contrast, TNFα and IL6 concentrations in large intestine extracts were significantly reduced after BX471 treatment as compared with large intestine extracts from vehicle-treated animals. This suggests that CCR1 blockade exerted anti-inflammatory effects, which prevented increases in W/D ratios to the level observed in vehicle treated animals.

Furthermore, it is suggested that blockade of CCR1 and of CCR2 can reduce the magnitude of the release of inflammatory molecules which contribute to the complex inflammation cascades that regulate organ function during shock phases. Thus, the finding that CCL5 concentrations were decreased in vehicle-treated animals after 6 h of resuscitation could be explained by transient increases in CCL5 levels during early resuscitation periods with subsequent decreases of CCL5 levels after longer time periods. Nevertheless, the findings that BX471 and INCB3284 reduced hemorrhage and resuscitation-associated increases in TNFα and IL-6 concentrations, as well as increases of the sum of inflammation markers that showed elevated concentrations at the end of the resuscitation period, support the concept that these drugs can attenuate the systemic inflammatory response during resuscitation after hemorrhage. Furthermore, measurements of systemic inflammation markers suggested that BX471 and INCB3284 attenuate release of TNFα, IL-6, IFNγ, IL1α, IL1α, CCL2 and CCL3, and enhance release of CCL5 during resuscitation.

### Therapeutically Effective Amount

It was previously reported that administration of 0.5 μmol/kg BX471 and of 5 μmol/kg INCB3284 provide fluid sparing effects during resuscitation after 30 min of hemorrhage to a MAP of 30 mmHg when animals were resuscitated for up to 3 hours and 4.5 hours, respectively (Id.). In some examples, the methods disclosed herein comprise a CCR1 inhibitor, such as, for example, BX471, CCX354, BMS-817399, MLN3897, CP-481,715, pharmaceutically acceptable salts or derivatives thereof, alone or in any combination thereof. In some examples, the CCR1 inhibitor can be administered in a therapeutically effective amount, which for example can be from about 0.1 to 10 μmol/kg of the patient (e.g., 0.1-9 μmol/kg, 9 μmol/kg-10 μmol/kg, 0.1 μmol/kg-8 μmol/kg, 8 μmol/kg-10 μmol/kg, 0.1 μmol/kg-7 μmol/kg, 7 μmol/kg-10 μmol/kg, 0.1 μmol/kg-6 μmol/kg, 6 μmol/kg-10 μmol/kg, 0.1 μmol/kg-5 μmol/kg, 5 μmol/kg-10 μmol/kg, 0.1 μmol/kg-4 μmol/kg, 4 μmol/kg-8 μmol/kg, 4 μmol/kg-10 μmol/kg, 0.1 μmol/kg-3 μmol/kg, 3 μmol/kg-6 μmol/kg, 6 μmol/kg-9 μmol/kg, 3 μmol/kg-10 μmol/kg, 0.1 μmol/kg-2 μmol/kg, 2 μmol/kg-4 μmol/kg, 4 μmol/kg-6 μmol/kg, 6 μmol/kg-8 μmol/kg, 2 μmol/kg-10 μmol/kg, 0.1 μmol/kg-1 μmol/kg, 1 μmol/kg-2 μmol/kg, 2 μmol/kg-3 μmol/kg, 3 μmol/kg-4 μmol/kg, 4 μmol/kg-5 μmol/kg, 5 μmol/kg-6 μmol/kg, 6 μmol/kg-7 μmol/kg, 7 μmol/kg-8 μmol/kg, 8 μmol/kg-9 μmol/kg, or 1 μmol/kg-10 μmol/kg). In some examples, the CCR1 inhibitor can be administered in a therapeutically effective amount, which for example can be from about 0.1 to 1 μmol/kg of the patient (e.g., 0.1 μmol/kg-0.9 μmol/kg, 0.9 μmol/kg-1 μmol/kg, 0.1 μmol/kg-0.8 μmol/kg, 0.8 μmol/kg-1 μmol/kg, 0.1 μmol/kg-0.7 μmol/kg, 0.7 μmol/kg-1 μmol/kg, 0.1 μmol/kg-0.6 μmol/kg, 0.6 μmol/kg-1 μmol/kg, 0.1 μmol/kg-0.5 μmol/kg, 0.5 μmol/kg-1 μmol/kg, 0.1 μmol/kg-0.4 μmol/kg, 0.4 μmol/kg-0.8 μmol/kg, 0.4 μmol/kg-1 μmol/kg, 0.1 μmol/kg-0.3 μmol/kg, 0.3 μmol/kg-0.6 μmol/kg, 0.6 μmol/kg-0.9 μmol/kg, 0.3 μmol/kg-1 μmol/kg, 0.1 μmol/kg-0.2 μmol/kg, 0.2 μmol/kg-0.3 μmol/kg, 0.3 μmol/kg-0.4 μmol/kg, 0.4 μmol/kg-0.5 μmol/kg, 0.5 μmol/kg-0.6 μmol/kg, 0.6 μmol/kg-0.7 μmol/kg, 0.7 μmol/kg-0.8 μmol/kg, 0.8 μmol/kg-0.9 μmol/kg, or 0.2 μmol/kg-1 μmol/kg). In some examples, the therapeutically effective amount can be about 0.5 μmol/kg. In some examples, the therapeutically effective amount can be about 5 μmol/kg.

In some examples, the methods disclosed herein comprise BX471 or a pharmaceutically acceptable salt thereof. In some examples, BX471 or a pharmaceutically acceptable salt thereof can be administered in a therapeutically effective amount, which for example can be from about 0.1 to 10 μmol/kg of the patient (e.g., 0.1-9 μmol/kg, 9 μmol/kg-10 μmol/kg, 0.1 μmol/kg-8 μmol/kg, 8 μmol/kg-10 μmol/kg, 0.1 μmol/kg-7 μmol/kg, 7 μmol/kg-10 μmol/kg, 0.1 μmol/kg-6 μmol/kg, 6 μmol/kg-10 μmol/kg, 0.1 μmol/kg-5 μmol/kg, 5 μmol/kg-10 μmol/kg, 0.1 μmol/kg-4 μmol/kg, 4 μmol/kg-8 μmol/kg, 4 μmol/kg-10 μmol/kg, 0.1 μmol/kg-3 μmol/kg, 3 μmol/kg-6 μmol/kg, 6 μmol/kg-9 μmol/kg, 3 μmol/kg-10 μmol/kg, 0.1 μmol/kg-2 μmol/kg, 2 μmol/kg-4 μmol/kg, 4 μmol/kg-6 μmol/kg, 6 μmol/kg-8 μmol/kg, 2 μmol/kg-10 μmol/kg, 0.1 μmol/kg-1 μmol/kg, 1 μmol/kg-2 μmol/kg, 2 μmol/kg-3 μmol/kg, 3 μmol/kg-4 μmol/kg, 4 μmol/kg-5 μmol/kg, 5 μmol/kg-6 μmol/kg, 6 μmol/kg-7 μmol/kg, 7 μmol/kg-8 μmol/kg, 8 μmol/kg-9 μmol/kg, or 1 μmol/kg-10 μmol/kg). In some examples, BX471 or a pharmaceutically acceptable salt thereof can be administered in a therapeutically effective amount, which for example can be from about 0.1 to 1 μmol/kg of the patient (e.g., 0.1 μmol/kg-0.9 μmol/kg, 0.9 μmol/kg-1 μmol/kg, 0.1 μmol/kg-0.8 μmol/kg, 0.8 μmol/kg-1 μmol/kg, 0.1 μmol/kg-0.7 μmol/kg, 0.7 μmol/kg-1 μmol/kg, 0.1 μmol/kg-0.6 μmol/kg, 0.6 μmol/kg-1 μmol/kg, 0.1 μmol/kg-0.5 μmol/kg, 0.5 μmol/kg-1 μmol/kg, 0.1 μmol/kg-0.4 μmol/kg, 0.4 μmol/kg-0.8 μmol/kg, 0.4 μmol/kg-1 μmol/kg, 0.1 μmol/kg-0.3 μmol/kg, 0.3 μmol/kg-0.6 μmol/kg, 0.6 μmol/kg-0.9 μmol/kg, 0.3 μmol/kg-1 μmol/kg, 0.1 μmol/kg-0.2 μmol/kg, 0.2 μmol/kg-0.3 μmol/kg, 0.3 μmol/kg-0.4 μmol/kg, 0.4 μmol/kg-0.5 μmol/kg, 0.5 μmol/kg-0.6 μmol/kg, 0.6 μmol/kg-0.7 μmol/kg, 0.7 μmol/kg-0.8 μmol/kg, 0.8 μmol/kg-0.9 μmol/kg, or 0.2 μmol/kg-1 μmol/kg). In some examples, the therapeutically effective amount can be about 0.5 μmol/kg. In some examples, the therapeutically effective amount can be about 5 μmol/kg.

In some examples, the methods disclosed herein comprise a CCR1 inhibitor and a CCR inhibitor, such as, for example, a CCR2 inhibitor, a CCR3 inhibitor, a CCR5 inhibitor, or any combination thereof. In some examples, the CCR2 inhibitor, the CCR3 inhibitor, the CCR5 inhibitor, or combinations thereof can be administered in a therapeutically effective amount, which for example can be from about 0.1 to 10 μmol/kg of the patient (e.g., 0.1-9 μmol/kg, 9 μmol/kg-10 μmol/kg, 0.1 μmol/kg-8 μmol/kg, 8 μmol/kg-10 μmol/kg, 0.1 μmol/kg-7 μmol/kg, 7 μmol/kg-10 μmol/kg, 0.1 μmol/kg-6 μmol/kg, 6 μmol/kg-10 μmol/kg, 0.1 μmol/kg-5 μmol/kg, 5 μmol/kg-10 μmol/kg, 0.1 μmol/kg-4 μmol/kg, 4 μmol/kg-8 μmol/kg, 4 μmol/kg-10 μmol/kg, 0.1 μmol/kg-3 μmol/kg, 3 μmol/kg-6 μmol/kg, 6 μmol/kg-9 μmol/kg, 3 μmol/kg-10 μmol/kg, 0.1 μmol/kg-2 μmol/kg, 2 μmol/kg-4 μmol/kg, 4 μmol/kg-6 μmol/kg, 6 μmol/kg-8 μmol/kg, 2 μmol/kg-10 μmol/kg, 0.1 μmol/kg-1 μmol/kg, 1 μmol/kg-2 μmol/kg, 2 μmol/kg-3 μmol/kg, 3 μmol/kg-4 μmol/kg, 4 μmol/kg-5 μmol/kg, 5 μmol/kg-6 μmol/kg, 6 μmol/kg-7 μmol/kg, 7 μmol/kg-8 μmol/kg, 8 μmol/kg-9 μmol/kg, or 1 μmol/kg-10 μmol/kg). In some examples, the CCR2 inhibitor, the CCR3 inhibitor, the CCR5 inhibitor, or combinations thereof can be administered in a therapeutically effective amount, which for example can be from about 0.1 to 1 μmol/kg of the patient (e.g., 0.1 μmol/kg-0.9 μmol/kg, 0.9 μmol/kg-1 μmol/kg, 0.1 μmol/kg-0.8 μmol/kg, 0.8 μmol/kg-1 μmol/kg, 0.1 μmol/kg-0.7 μmol/kg, 0.7 μmol/kg-1 μmol/kg, 0.1 μmol/kg-0.6 μmol/kg, 0.6 μmol/kg-1 μmol/kg, 0.1 μmol/kg-0.5 μmol/kg, 0.5 μmol/kg-1 μmol/kg, 0.1 μmol/kg-0.4 μmol/kg, 0.4 μmol/kg-0.8 μmol/kg, 0.4 μmol/kg-1 μmol/kg, 0.1 μmol/kg-0.3 μmol/kg, 0.3 μmol/kg-0.6 μmol/kg, 0.6 μmol/kg-0.9 μmol/kg, 0.3 μmol/kg-1 μmol/kg, 0.1 μmol/kg-0.2 μmol/kg, 0.2 μmol/kg-0.3 μmol/kg, 0.3 μmol/kg-0.4 μmol/kg, 0.4 μmol/kg-0.5 μmol/kg, 0.5 μmol/kg-0.6 μmol/kg, 0.6 μmol/kg-0.7 μmol/kg, 0.7 μmol/kg-0.8 μmol/kg, 0.8 μmol/kg-0.9 μmol/kg, or 0.2 μmol/kg-1 μmol/kg). In some examples, the therapeutically effective amount can be about 0.5 μmol/kg. In some examples, the therapeutically effective amount can be about 5 μmol/kg.

In some examples, the methods disclosed herein comprise a CCR2 inhibitor (e.g., INCB3284 or a pharmaceutically acceptable salt thereof). In some examples, the CCR2 inhibitor can be administered in a therapeutically effective amount, which for example can be from about 0.1 to 10 μmol/kg of the patient (e.g., 0.1-9 μmol/kg, 9 μmol/kg-10 μmol/kg, 0.1 μmol/kg-8 μmol/kg, 8 μmol/kg-10 μmol/kg, 0.1 μmol/kg-7 μmol/kg, 7 μmol/kg-10 μmol/kg, 0.1 μmol/kg-6 μmol/kg, 6 μmol/kg-10 μmol/kg, 0.1 μmol/kg-5 μmol/kg, 5 μmol/kg-10 μmol/kg, 0.1 μmol/kg-4 μmol/kg, 4 μmol/kg-8 μmol/kg, 4 μmol/kg-10 μmol/kg, 0.1 μmol/kg-3 μmol/kg, 3 μmol/kg-6 μmol/kg, 6 μmol/kg-9 μmol/kg, 3 μmol/kg-10 μmol/kg, 0.1 μmol/kg-2 μmol/kg, 2 μmol/kg-4 μmol/kg, 4 μmol/kg-6 μmol/kg, 6 μmol/kg-8 μmol/kg, 2 μmol/kg-10 μmol/kg, 0.1 μmol/kg-1 μmol/kg, 1 μmol/kg-2 μmol/kg, 2 μmol/kg-3 μmol/kg, 3 μmol/kg-4 μmol/kg, 4 μmol/kg-5 μmol/kg, 5 μmol/kg-6 μmol/kg, 6 μmol/kg-7 μmol/kg, 7 μmol/kg-8 μmol/kg, 8 μmol/kg-9 μmol/kg, or 1 μmol/kg-10 μmol/kg). In some examples, the CCR2 inhibitor can be administered in a therapeutically effective amount, which for example can be from about 0.1 to 1 μmol/kg of the patient (e.g., 0.1 μmol/kg-0.9 μmol/kg, 0.9 μmol/kg-1 μmol/kg, 0.1 μmol/kg-0.8 μmol/kg, 0.8 μmol/kg-1 μmol/kg, 0.1 μmol/kg-0.7 μmol/kg, 0.7 μmol/kg-1 μmol/kg, 0.1 μmol/kg-0.6 μmol/kg, 0.6 μmol/kg-1 μmol/kg, 0.1 μmol/kg-0.5 μmol/kg, 0.5 μmol/kg-1 μmol/kg, 0.1 μmol/kg-0.4 μmol/kg, 0.4 μmol/kg-0.8 μmol/kg, 0.4 μmol/kg-1 μmol/kg, 0.1 μmol/kg-0.3 μmol/kg, 0.3 μmol/kg-0.6 μmol/kg, 0.6 μmol/kg-0.9 μmol/kg, 0.3 μmol/kg-1 μmol/kg, 0.1 μmol/kg-0.2 μmol/kg, 0.2 μmol/kg-0.3 μmol/kg, 0.3 μmol/kg-0.4 μmol/kg, 0.4 μmol/kg-0.5 μmol/kg, 0.5 μmol/kg-0.6 μmol/kg, 0.6 μmol/kg-0.7 μmol/kg, 0.7 μmol/kg-0.8 μmol/kg, 0.8 μmol/kg-0.9 μmol/kg, or 0.2 μmol/kg-1 μmol/kg). In some examples, the therapeutically effective amount can be about 0.5 μmol/kg. In some examples, the therapeutically effective amount can be about 5 μmol/kg.

### Administration

In some examples, the CCR1 inhibitor of the methods disclosed herein can be administered to the patient separately from the resuscitation fluid. For example, the CCR1 inhibitor can be administered in a separate composition from the resuscitation fluid. In some examples, the CCR1 inhibitor can be administered before, concurrently, or after administering the resuscitation fluid. In some examples, the CCR1 inhibitor can be a component of the resuscitation fluid.

In some examples, BX471 or a pharmaceutically acceptable salt thereof of the methods disclosed herein can be administered to the patient separately from the resuscitation fluid. For example, BX471 or a pharmaceutically acceptable salt thereof can be administered in a separate composition from the resuscitation fluid. In some examples, BX471 or a pharmaceutically acceptable salt thereof can be administered before, concurrently, or after administering the resuscitation fluid. In some examples, BX471 or a pharmaceutically acceptable salt thereof can be a component of the resuscitation fluid.

In some examples, the CCR inhibitor such as the CCR2 inhibitor, CCR3 inhibitor, CCR5 inhibitor, or combinations thereof of the methods disclosed herein can be administered to the patient separately from the resuscitation fluid. For example, the CCR2 inhibitor, the CCR3 inhibitor, the CCR5 inhibitor, or combinations thereof can be administered in a separate composition from the resuscitation fluid. In some examples, the CCR2 inhibitor, the CCR3 inhibitor, the CCR5 inhibitor, or combinations thereof can be administered before, concurrently, or after administering the resuscitation fluid. In some examples, the CCR2 inhibitor, the CCR3 inhibitor, the CCR5 inhibitor, or combinations thereof can be a component of the resuscitation fluid.

In some examples, BX471 or a pharmaceutically acceptable salt thereof and INCB3284 or a pharmaceutically acceptable salt thereof of the methods disclosed herein can be administered to the patient separately from the resuscitation fluid. For example, BX471 or a pharmaceutically acceptable salt thereof and INCB3284 or a pharmaceutically acceptable salt thereof can be administered in a separate composition from the resuscitation fluid. In some examples, BX471 or a pharmaceutically acceptable salt thereof and INCB3284 or a pharmaceutically acceptable salt thereof can be administered before, concurrently, or after administering the resuscitation fluid. In some examples, BX471 or a pharmaceutically acceptable salt thereof and INCB3284 or a pharmaceutically acceptable salt thereof can be a component of the resuscitation fluid.

In some examples, the CCR1 inhibitor of the methods disclosed herein can be administered in one or multiple doses, such as one or at least two doses. In some examples, BX471 or a pharmaceutically acceptable salt thereof of the methods disclosed herein can be administered in one or multiple doses, such as one or at least two doses. In some examples, the CCR inhibitor such as the CCR2 inhibitor, CCR3 inhibitor, CCR5 inhibitor, or combinations thereof of the methods disclosed herein can be administered in one or multiple doses, such as one or at least two doses. In some examples, BX471 or a pharmaceutically acceptable salt thereof and INCB3284 or a pharmaceutically acceptable salt thereof of the methods disclosed herein can be administered in one or multiple doses, such as one or at least two doses.

In some examples, the CCR1 inhibitor can be administered continuously as a component of the resuscitation fluid. In some examples, BX471 or a pharmaceutically acceptable salt thereof can be administered continuously as a component of the resuscitation fluid. In some examples, the CCR1 inhibitor and the CCR inhibitor such as the CCR2 inhibitor, CCR3 inhibitor, CCR5 inhibitor, or combinations thereof can be administered continuously as a component of the resuscitation fluid. In some examples, BX471 or a pharmaceutically acceptable salt thereof and INCB3284 or a pharmaceutically acceptable salt thereof can be administered continuously as a component of the resuscitation fluid.

In some examples, the CCR1 inhibitor of the methods disclosed herein can be administered to the patient as an intravenous (iv) bolus. In some examples, BX471 or a pharmaceutically acceptable salt thereof of the methods disclosed herein can be administered to the patient as an intravenous (iv) bolus. In some examples, a CCR inhibitor such as the CCR2 inhibitor, CCR3 inhibitor, CCR5 inhibitor, or combinations thereof of the methods disclosed herein can be administered to the patient as an intravenous (iv) bolus. In some examples, BX471 or a pharmaceutically acceptable salt thereof and INCB3284 or a pharmaceutically acceptable salt thereof of the methods disclosed herein can be administered to the patient as an intravenous (iv) bolus.

In some examples, the methods disclosed herein further include administering a bolus of an iso or hypotonic solution to maintain hemodynamic parameters of stable circulation.

For example, the bolus of an iso or hypotonic solution can be administered to achieve a mean arterial pressure (MAP) greater than about 60 mm Hg or to achieve a systolic blood pressure of greater than about 90 mm Hg. In some examples, the bolus of an iso or hypotonic solution can be administered to achieve a MAP of from about 60 mm Hg-110 mm Hg (e.g., from about 60 mm Hg-100 mm Hg, 100 mm Hg-110 mm Hg, 60 mm Hg-90 mm Hg, 90 mm Hg-110 mm Hg, 60 mm Hg-80 mm Hg 80 mm Hg-100 mm Hg, 80 mm Hg-110 mm Hg, 60 mm Hg-70 mm Hg, 70 mm Hg-80 mm Hg, 80 mm Hg-90 mm Hg, or 70 mm Hg-110 mm Hg). In some examples, the bolus of an iso or hypotonic solution can be administered to achieve a systolic blood pressure of from about 100-120 mm Hg (e.g., from about 100-110 mm Hg, 110-120 mm Hg, 100-115 mm Hg, or 115-120 mm Hg).

In some examples, the resuscitation fluid can be an iso or hypertonic salt solution, although normal saline or other similar isotonic crystalloid solutions can be used. For example, isotonic fluid replacement solutions can include solutions where isotonic crystalloid solutions are mixed with macromolecular solutions of plasma proteins or synthesized molecules with similar oncotic properties (colloids) including albumin, dextran, hetastarch or polygelatin in NaCl. In some examples, the resuscitation fluid can be an isotonic crystalloid fluid, such as, for example, 0.9% sodium chloride or normal saline, 0.45% sodium chloride or half normal saline, 3% sodium chloride or hypertonic saline, Ringer's solution, lactated Ringer's solution, dextrose 5% in water (D5W), or acetate-based infusion fluids such as PlasmaLyte™, Hartmann's™, or acetated Ringer's solution. In some examples, the resuscitation fluid can be Ringer's solution or lactated Ringer's solution. In general, any resuscitation fluid can be used. For example, in some cases the resuscitation fluid can comprise whole blood.

Resuscitation fluids have typically been used a dosage rate of about 3 times the amount of blood loss. Stated differently, for every liter of blood loss, a resuscitation fluid treatment required 3 liters of resuscitation fluid. Recommended continued treatment is based on the observed response to the initial fluid therapy. American College of Surgeons, 154, 585-588, (1987). As a general rule, guidelines are based on the “three for one” rule. This is based on the long-standing empirical observation that most hemorrhagic shock patients require up to 300 mL of electrolyte solution for each 100 mL of blood lost. The present methods, however, are intended to use resuscitation fluids in dosages less than the total amount of blood loss. Thus, disclosed herein are methods whereby the resuscitation fluid is administered in an amount that is less than three times the amount of fluid lost by the patient, e.g., 2.5 times, 2 times, 1.5 times, the same, or less than the amount of fluid (e.g., blood), lost by the patient. In some examples, disclosed herein are methods whereby the resuscitation fluid is administered in an amount that is from about the same to 1.5 times the amount of fluid (e.g., blood), lost by the patient. In some examples, disclosed herein are methods whereby the resuscitation fluid is administered in an amount that is about the same as the amount of fluid (e.g., blood), lost by the patient. In some examples, disclosed herein are methods whereby the resuscitation fluid is administered in an amount that is about 1.1 times the amount of fluid (e.g., blood), lost by the patient.

It was previously reported that administration of 0.5 μmol/kg BX471 and of 5 μmol/kg INCB3284 provide fluid sparing effects during resuscitation after 30 min of hemorrhage to a MAP of 30 mmHg when animals were resuscitated for up to 3 hours and 4.5 hours, respectively (Id.). The findings suggest that such fluid sparing effects of the same doses of BX471 and INCB3284 were detectable during resuscitation periods of up to 6 hours. Furthermore, administration of BX471 reduced fluid requirements by approximately 70%, as compared with vehicle-treated animals. Because the volume of resuscitation fluid was equal to the hemorrhage volume in BX471-treated animals, any further reduction of fluid requirements could not be possible, which could have masked potential synergistic or additive effects of co-administration of BX471 and INCB3284.

The resuscitation fluid, the CCR1 inhibitor (e.g., BX471 or a pharmaceutically acceptable salt thereof), and the CCR inhibitor such as the CCR2 inhibitor (e.g., INCB3284 or a pharmaceutically acceptable salt thereof), CCR3 inhibitor, CCR5 inhibitor, or combinations as disclosed herein can be administered, alone or in any combination, to the patient by intraperitoneal injection, intravenous administration, subcutaneous administration, sublingual administration, inhalation, oral administration, topical application to a blood vessel, or coating of a device to be placed within the subject.

In some examples, the patient can be administered the resuscitation fluid for up to about 6 hours, e.g. 4 hours or 3.5 hours. In some examples, the patient can be administered the resuscitation fluid for greater than about 4 hours, e.g., 4 hours, 6 hours, or 12 hours.

The disclosed methods can further comprise the additional administration of other active compounds. These additional active compounds include but are not limited to antibiotics, analgesics, anti-inflammatory drugs, antihistamines, sedatives, corticosteroids, electrolytes, gastro-intestinal drugs, muscle relaxants, nutritional agents, vitamins, stimulants, and antiviral agents.

### EXAMPLES

The following examples are put forth so as to provide those of ordinary skill in the art with a complete disclosure and description of how the compounds and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

Drugs. BX471 was purchased from Tocris, Bio-Techne Corporation (Minneapolis, MN, USA).

Male Sprague-Daley rats (320-400g) were obtained from Envigo (Indianapolis, IN, USA). General anesthesia was first induced utilizing an isoflurane-soaked gauze and a bell jar. Once anesthetized, the animal was transferred to the procedural area where sedation was maintained at 2.6% isoflurane utilizing nose cone inhalation with the SomnoSuite small animal anesthesia system (Kent Scientific Corporation, Torrington, CT, USA). This achieved a level of sedation such that the animal no longer responded to noxious stimuli but still maintained spontaneous respiration. Utilizing a direct cut down method, the left femoral artery and right femoral vein were isolated and cannulated with a 24-gauge and 26-gauge catheter, respectively. The arterial line was used for hemodynamic monitoring and blood withdrawal and the venous line was used for drug and fluid administration. Following cannulation, isoflurane was reduced to 1.7% and the animals were monitored for a 10-minute period prior to starting the experiment to ensure hemodynamic and respiratory stability. Hemodynamics were monitored continuously with the Surgivet invasive blood pressure monitor (Med-Electronics, Beltsville, MD, USA). Two series were completed with experiments performed in an alternating fashion. In both series, hemodynamics were monitored continuously and recorded at 2 min intervals. The level of sedation was maintained until the animal expired or was euthanized at the conclusion of the experiment.

Series 1: Rats were hemorrhaged to a mean arterial blood pressure (MAP) of 30 mmHg for 30 min. At the end of this shock period (t=30 min), animals received either 1 mL of lactated ringer's solution (LR) (=control group, n=5), 1 mL of 0.05 μmol/kg BX471 (n=5) or 1 mL of 0.5 μmol/kg BX471 (n=4) in LR as a bolus injection. Thereafter, all animals received 1 mL LR boluses of LR until either systolic blood pressure (SBP) recovered to 90 mmHg or MAP recovered to 60 mmHg. Subsequent 1-0.5 mL boluses of LR were administered as needed to maintain this blood pressure goal for the entirety of the 60 min resuscitation period. Blood samples for the analyses of blood gases and laboratory parameters were collected at the beginning of hemorrhage (t=0 min, the end of hemorrhage (t=30 min), and at the end of fluid resuscitation (t=90 min). At the conclusion of the experiment (t=90 min), surviving animals were euthanized (5% isoflurane, bilateral pneumothorax, ventriculotomy).

Series 2: Rats were hemorrhaged and resuscitated as in series 1 for 180 min. At the end of the shock period (t=30 min), animals received 1 mL of LR (=control, n=8) or 1 mL of 0.5 μmol/kg BX471 (n=7) in LR as a bolus injection. Blood samples were collected at the beginning of hemorrhage (t=0 min), the end of hemorrhage (t=30 min, at the midpoint (t=120 min) and at the end of fluid resuscitation (t=210 min) for the analyses of blood gases and laboratory parameters. Serum samples were collected at t=0 min and t=210 min and stored at −80° C. for subsequent analyses. At the conclusion of the experiment (t=210 min), surviving animals were euthanized (5% isoflurane, bilateral pneumothorax, ventriculotomy). Tissue samples of lung, small intestine, and large intestine were collected and used for the calculation of wet-weight to dry-weight (W/D) ratios or stored at −80° C. for further analyses.

Arterial Blood Gases and Laboratory Parameters. Arterial blood gases, electrolytes, creatinine, lactate, hemoglobin, and hematocrit were analyzed in both series at the time points outlined above with the use of the Element point of care veterinary blood gas, electrolyte, and critical care analyzer (Cuattro Veterinary USA, Loveland, CO, USA).

Tissue extracts. Tissue samples (small and large intestine, lung) were weighed and placed in cold PBS at a 1:4 weight to volume ratio. Samples were homogenized with the Biospec Products Tissue-Tearor (Bartlesville, OK, USA) over four 15 s intervals with 15 s of rest in between. The homogenates were then centrifuged at 15,000 g for 20 min, the supernatants (=tissue extracts) were aliquoted and used for further analyses.

Protein measurements. The colorimetric Bio-Rad Protein Assay Kit (Hercules, CA, USA) was used to measure total protein concentrations in serum samples and tissue homogenates according to the manufacturer's instructions. Bovine serum albumin was used as protein standard.

Measurements of Chemokine Concentrations. Serum levels of CCL3, CCL4, CCL5 and CCL7 were measured with commercially available rat enzyme-linked immunosorbent assays (ELISA, Boster Bio, Pleasanton, CA, USA) according to the manufacturer's instructions. Chemokine concentrations were calculated per mg of total protein.

Measurements of myeloperoxidase (APO) activity. Relative myeloperoxidase (MPO) activity was measured in tissue homogenates utilizing the commercially available fluorescent EnzChek Myeloperoxidase (MPO) Activity Assay Kit according to manufacturer's instructions (Thermo Fisher Scientific, Waltham, MA, USA). MPO activity was calculated as relative fluorescence units (RFU)/mg of protein/min.

Multiplex ELISA. The commercially available Rat Inflammation ELISA Colorimetric Strip from Signosis (Santa Clara, CA, USA) was used to measure relative concentrations of tumor necrosis factor α (TNFα), interleukin 6 (IL6), interferon γ (IFNγ), interleukin 1α (IL1α), IL-1β, CCL2, CCL3 and CCL5 in tissue homogenates according to manufacturer's instructions. Relative concentrations of the analytes were expressed as optical density (OD) measured at λ450 nm/mg of protein.

Wet-weight to dry-weight ratios. The ratio of the tissue wet-weight to dry-weight (W/D ratio) was determined gravimetrically, as described (Id.).

Data analyses and statistics. Data are presented as mean±standard error (SE). Data were analyzed with Fisher's exact test, unpaired t-test and 2-way analysis of variance (ANOVA) with Tukey's multiple comparisons test, as appropriate. All analyses were calculated with the GraphPad Prism program version 10.3.1 (GraphPad Software, San Diego, CA, USA). A 2-tailed p<0.05 was considered significant.

Sprague-Dawley rats were hemorrhaged for 30 min, followed by fluid resuscitation to maintain blood pressure for 60 min (series 1) and 180 min (series 2). Series 1: animals received vehicle (n=5), 0.05 μmol/kg (n=5) or 0.5 μmol/kg (n=4) BX471 at t=30 min. Series 2: animals received vehicle (n=8) or 0.5 μmol/kg (n=4) BX471 at t=30 min. Hemodynamics, fluid requirements, blood gases and lactate were monitored. Serum concentrations of CCR1 ligands (CCL3/4/5/7) were determined at baseline and at the conclusion of the experiments. Tissue (small/large intestine, lung) wet/dry (W/D) weight ratios, lung myeloperoxidase activity and a panel of inflammation markers in tissue extracts were measured.

### Example 2: Results

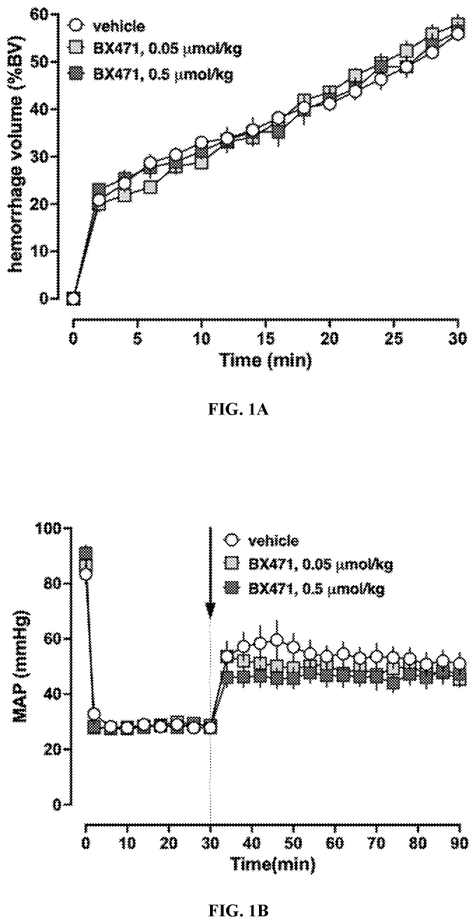

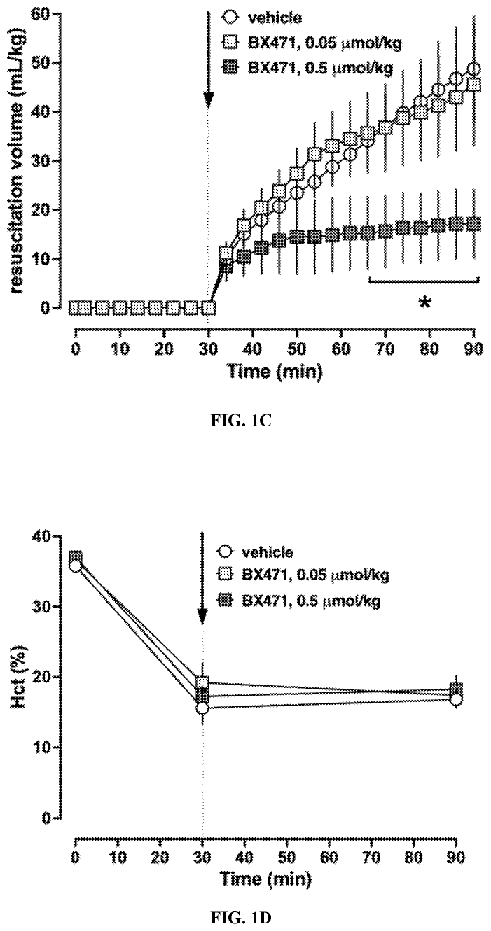

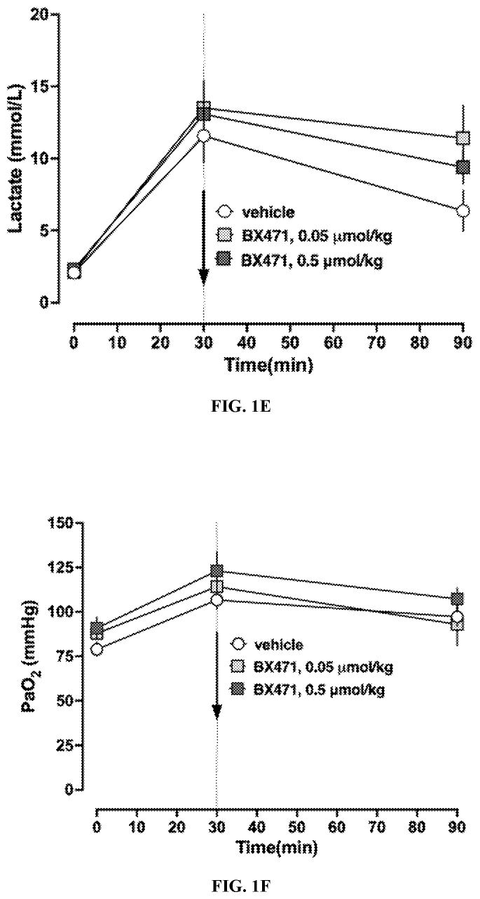

The object of this study was to evaluate the possible therapeutic potential of BX471 during resuscitation from hemorrhagic shock in rat models. The experimental design of the examples address early resuscitation strategies, e.g., a scenario comparable to pre-hospital and emergency room resuscitation in patients. As such, the experimental design reflects common clinical practice and adherence to evidence-based medicine. As indicated by the consistent increase in lactate concentrations to 10-15 mmol/L at the end of the hemorrhage period and the development of significant fluid requirements to maintain hemodynamics, the hemorrhagic shock and resuscitation model that was employed in the examples has reproducible pathophysiological consequences and thus can be used to place the current findings in the context of previous observations (Id.). To gain initial information on the effects of BX471 during resuscitation from hemorrhagic shock, animals were injected with a single intravenous (iv) bolus of vehicle, 0.05 μmol/kg or 0.5 μmol/kg BX471 at the end of the hemorrhage period, followed by crystalloid fluid resuscitation for 60 min (Series 1). There were no differences in any measured physiological parameters among groups at baseline. The hemorrhage volumes to achieve a MAP of 30 mmHg during the shock period were indistinguishable among groups, and all animals could be resuscitated to the target blood pressures (FIGS. 1A-1B). While the fluid requirements to achieve blood pressure targets during the resuscitation period were indistinguishable between animals treated with vehicle or 0.05 μmol/kg BX471, treatment with 0.5 μmol/kg BX471 significantly reduced fluid requirements by more than 60% (FIG. 1C). There were no significant differences in hematocrit values (FIG. 1D), blood lactate concentrations (FIG. 1E) or partial pressures of oxygen in arterial blood between the groups (FIG. 1F). All animals survived the observation period.

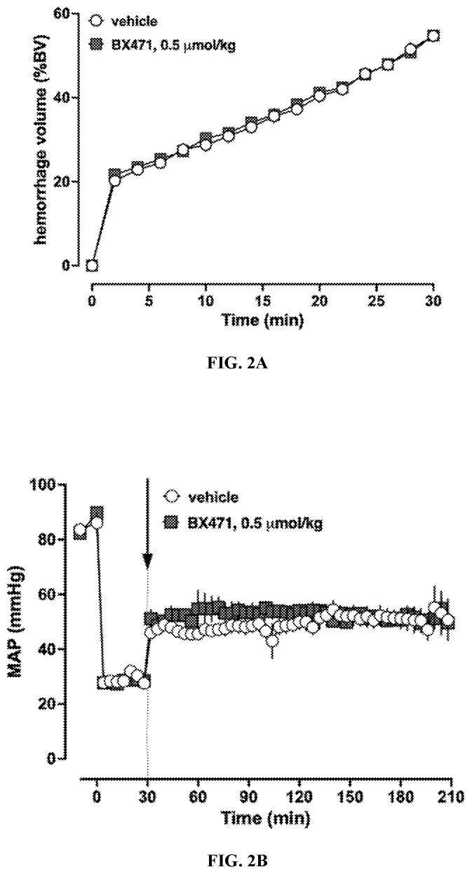

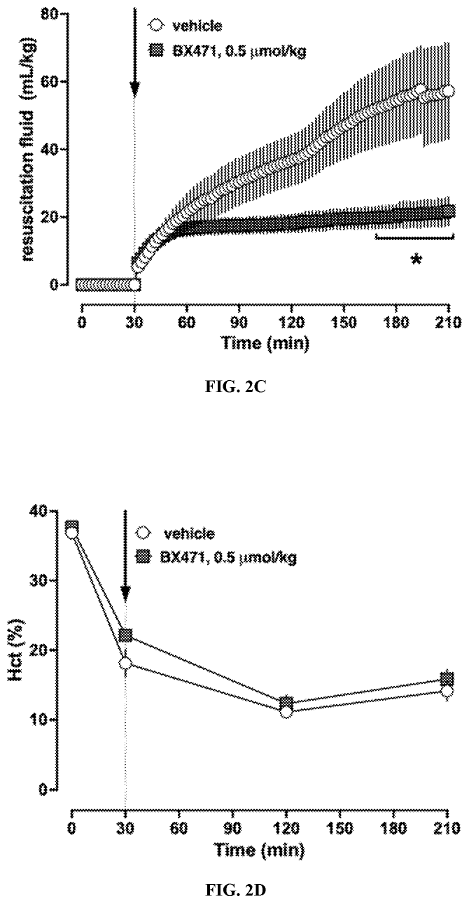

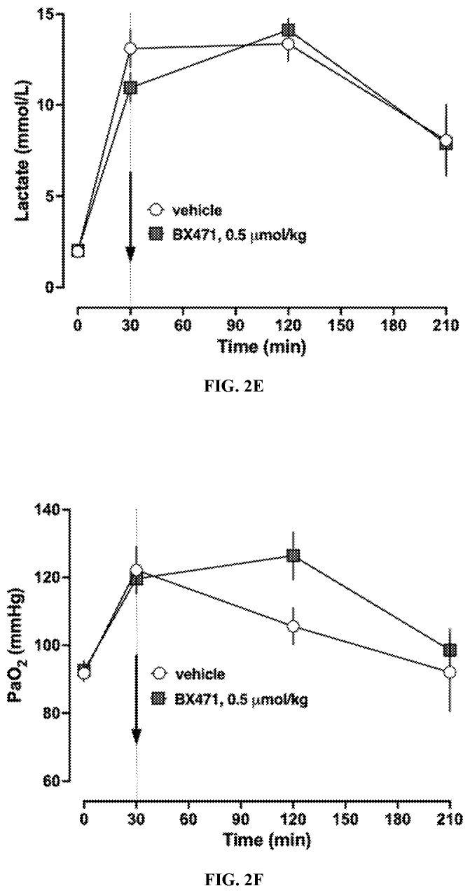

Based on these findings, a dose of 0.5 μmol/kg BX471 was selected to assess whether the observed fluid-sparing effects would persist over a resuscitation period of 3 h (Series 2). As in series 1, physiological parameters between vehicle and BX471-treated animals at baseline were indistinguishable and hemorrhage volumes to achieve a MAP of 30 mmHg were comparable (FIGS. 2A-2B). When compared with vehicle treated animals, treatment with BX471 significantly reduced fluid requirements to maintain MAP targets by more than 60% (FIG. 2C). Hematocrit values (FIG. 2D), lactate concentrations (FIG. 2E) and partial pressures of oxygen in arterial blood were indistinguishable between the groups (FIG. 2F). Seven of the 8 vehicle-treated animals and all BX471-treated animals survived the observation period (p>0.05).

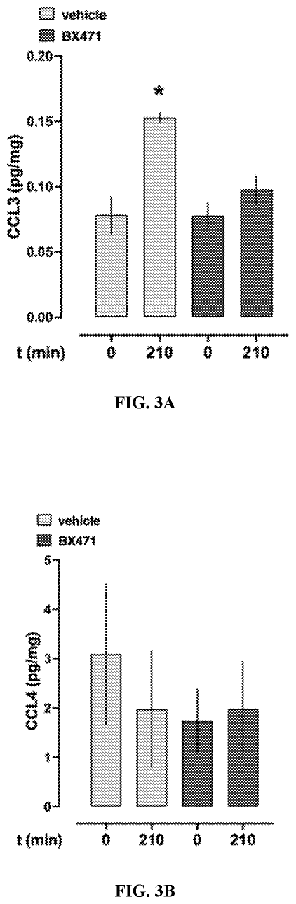

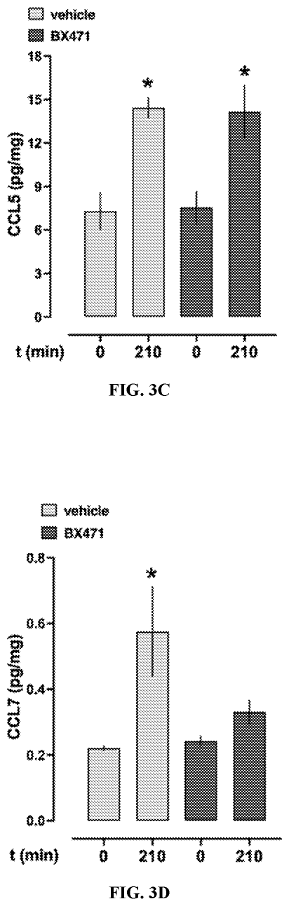

To assess whether endogenous CCR1 ligands are systemically released during resuscitation from hemorrhagic shock under the experimental conditions, serum levels of CCL3, CCL4, CCL5 and CCL7 were measured at baseline and at the conclusion of the experiment (FIGS. 3A-3D). In vehicle-treated animals, CCL3, CCL5 and CCL7 serum levels significantly increased from baseline to the end of the experiment (FIGS. 3A-3D), whereas CCL4 serum levels were not significantly altered (FIG. 3B). While CCL5 serum concentrations also significantly increased from baseline to the end of the experiment in BX471-treated animals (FIG. 3C), CCL3, CCL4 and CCL7 concentrations remained unchanged after BX471 treatment.

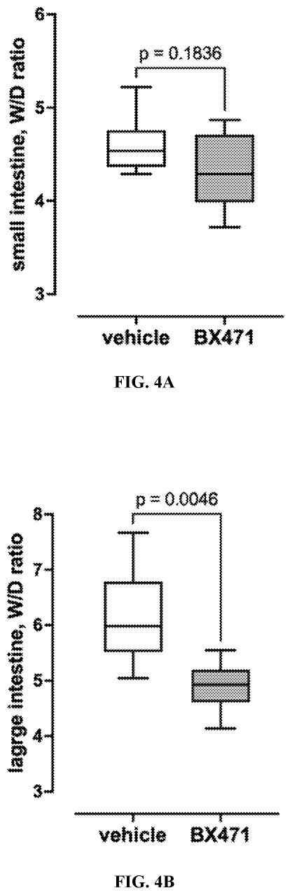

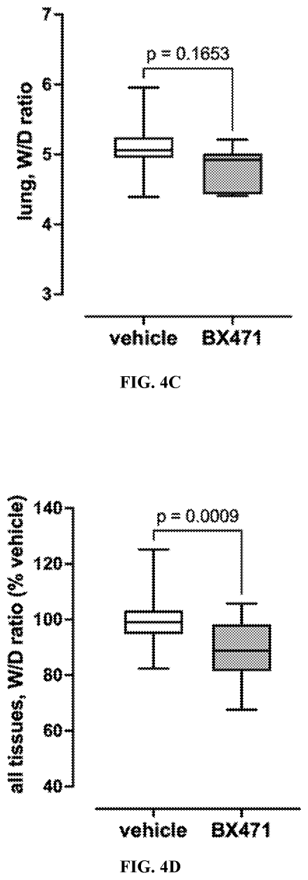

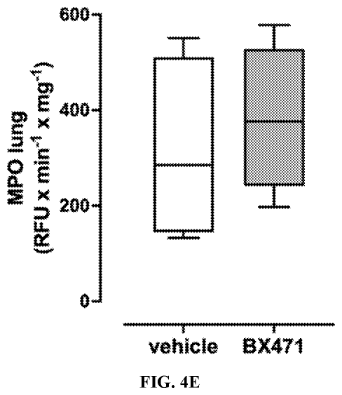

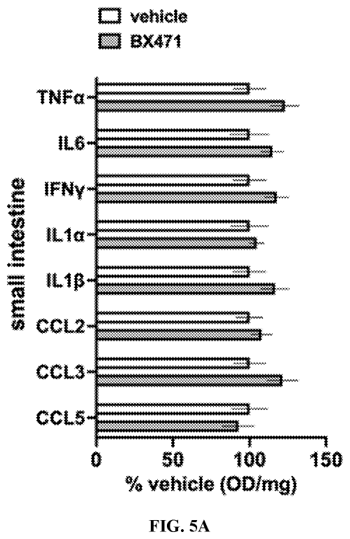

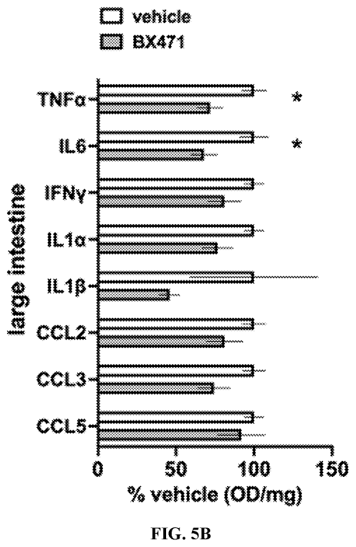

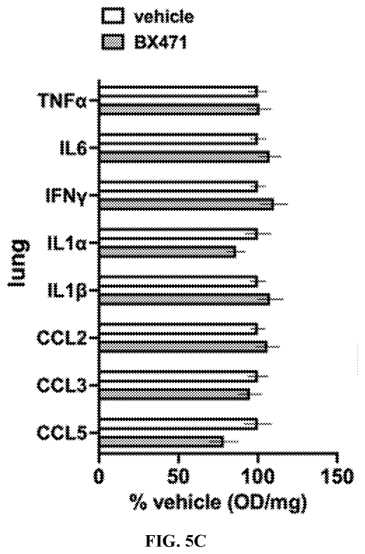

The W/D ratios measured in small and large intestine and in the lung are shown in FIGS. 4A-4D. Although W/D ratios of the small intestine (FIG. 4A) and the lung (FIG. 4C) were lower in BX471-treated animals than in vehicle treated animals, these differences did not reach statistical significance. BX471-treatment, however, significantly reduced W/D ratios of the large intestine (vehicle—6.12±0.29, BX471—4.9±0.16, p<0.05, FIG. 4B). When the relative changes of W/D ratios of the sum of all tissues (vehicle group=100%) were calculated as a parameter of the global tissue effects of the treatment, the W/D ratio was 89.5±230 2.2% with BX471 treatment (vehicle: 100±2.2%, p=0.0009 vs. BX471, FIG. 4D). Furthermore, there were no significant differences in MPO activity in lung extracts between the groups (FIG. 4E).

FIGS. 5A-5C shows the comparisons of the relative concentrations of a panel of inflammation markers in the tissue extracts. While significant differences were not observed for any of the inflammation markers in extracts from the small intestine and the lung (FIGS. 5A-5C), relative large intestine extract concentrations of TNFα and IL-6 were significantly lower with BX471 treatment, as compared with vehicle treatment (FIG. 5B).

All animals could be resuscitated to target blood pressures. Series 1: 0.5 μmol/kg BX471 reduced fluid requirements by more than 60% (p<0.05 vs. vehicle and 0.05 μmol/kg BX471). Series 2: Systemic CCL3/5/7 levels increased during the experiment (p<0.05). BX471-treatment reduced fluid requirements by more than 60% (p<0.05) and prevented increases in CCL3/7. W/D ratios of large intestine and of the sum of all tissues were lower with BX471 treatment (p<0.05). BX471-treatment reduced TNFα and IL6 concentrations in large intestine extracts (p<0.05).

### Example 3: Methods

Drugs. INCB3284 and BX471 were purchased from Tocris, Bio-Techne Corporation (Minneapolis, MN, USA).

Hemorrhagic shock models. Male Sprague-Dawley rats weighing between 330 and 400 g were obtained from Envigo (Indianapolis, IN, USA). General anesthesia was induced utilizing an isoflurane-soaked gauze and a bell jar. The animals were subsequently transferred to the procedural area where sedation was maintained using nosecone inhalation at a level of 2.6% isoflurane delivered by the SomnoSuite small animal anesthesia system (Kent Scientific Corporation, Torrington, CT, USA). Throughout the procedure, sedation was maintained at a level such that the animal was not responsive to noxious stimuli while still maintaining spontaneous respirations. The left femoral artery and right femoral vein were isolated utilizing a direct cutdown method and cannulated with a 24-gauge and 26-gauge catheter, respectively. The arterial line was used for continuous hemodynamic monitoring and as an access point for controlled hemorrhage. The venous line was used for drug and intravenous fluid administration. Following cannulation, isoflurane was reduced to 1.7% and the animal monitored for a period of 10 minutes to ensure stability before proceeding. Hemodynamics were monitored continuously throughout the experiment using the Surgivet invasive blood pressure monitor (Med-Electronics, Beltsville, MD, USA) and recorded at two-minute intervals during hemorrhage and five-minute intervals during the resuscitation or observation periods. In all experiments animals were continuously monitored and remained under general anesthesia until euthanasia or death, as defined by cessation of spontaneous respirations and MAP≤15 mmHg in all experiments.

Resuscitation series. Rats underwent controlled hemorrhage via the arterial line to a mean arterial blood pressure (MAP) of 30 mmHg for 30 min. At the end of the hemorrhage period (t=30 min), control animals (n=12) received lactated ringer's solution (=vehicle) delivered in 1 mL boluses via the femoral venous line until either systolic blood pressure (SBP) recovered to 90 mmHg or MAP recovered to 60 mmHg. Additional 0.5-1 mL boluses were administered as needed to maintain this blood pressure goal for the duration of the experiment. Experimental animals were hemorrhaged in an identical fashion and received either a 1 mL intravenous injection of 0.5 μmol/kg of BX471 (n=9), a 5 μmol/kg dose of INCB3284 (n=7), or a combination dose of 0.5 μmol/kg of BX471 plus 5 μmol/kg of INCB3284 (n=6) followed by additional LR boluses as needed to maintain hemodynamic goals. The doses of BX471 and INCB3284 were selected based on previous studies (Cook EA, Ogunsina O, Gao X, Majetschak M. The Chemokine (C-C Motif) Receptor 1 Antagonist BX471 Improves Fluid Resuscitation in Rat Models of Hemorrhagic Shock. Biomedicines. 2025; 13(5). Epub 2025/05/28. doi: 10.3390/biomedicines13051241. PubMed PMID: 40427068; PubMed Central PMCID: PMCPMC12109411; Id.). Epub 2022/05/28. doi: 10.1097/CCE.0000000000000701. PubMed PMID: 35620770; PubMed Central PMCID: PMCPMC9119637). For all groups, the experiment was concluded at the end of a 360-minute resuscitation period or at the time point the animal expired. Death was defined as cessation of spontaneous respirations and MAP≤15 mmHg. Blood samples were collected for blood gas and laboratory analysis at the beginning of hemorrhage (t=0 min), at the end of hemorrhage (t=30 min), midway through resuscitation (t=210 min), and at resuscitation end (t=390 min). Serum samples were collected at t=0 min and at the end of the experiment in animals who survived at least until t=330 min. Serum samples were stored at −80° C. for subsequent analysis. At the end of the experiment (t=390 min) or when death criteria had been met, animals were euthanized (5% isoflurane, bilateral pneumothorax, ventriculotomy).

Terminal hemorrhage series. Rats were hemorrhaged to a MAP of 30 mmHg for 45 min. At the end of this period, control animals (n=8) received a single 1 mL bolus of vehicle while experimental animals received either 0.5 μmol/kg BX471 (n=7) or 5 μmol/kg INCB3284 (n=5) delivered in a 1 mL bolus of vehicle. No additional fluid was administered in any group. Hemodynamics were observed continuously and recorded for a period of 180 minutes or until the animal expired, as defined above.

Arterial blood gases and laboratory parameters. Arterial blood gases, lactate, hemoglobin and hematocrit were measured using the Element point of care veterinary blood gas, electrolyte, and critical care analyzer (Cuattro Veterinary USA, Loveland, CO, USA).

Enzyme-linked immunosorbent assay (ELISA). Multiplex ELISA was performed measuring relative concentrations of tumor necrosis factor α (TNFα), interleukin 6 (IL6), interferon γ (IFNγ), IL1α, IL-1β, chemokine (C-C motif) ligand 2 (CCL2, also known as monocyte chemoattractant protein 1, MCP-1), CCL3 (also known as macrophage inflammatory protein 1α, MIP-1α) and CCL5 (also known as RANTES) in serum samples following manufacturer instructions using the commercially available Rat Inflammation ELISA Colorimetric Strip (Signosis, Santa Clara, CA, USA). Relative concentrations of the analytes were expressed as optical density (OD) measured at λ 450 nm/mg of protein.

Protein measurements. Total protein concentrations in serum samples were measured with the Thermo Scientific NanoDrop One Spectrophotometer (Waltham, MA, USA) according to the manufacturer's instructions.

Data analyses and statistics. The GraphPad Prism program version 10.3.1 (GraphPad Software, San Diego, CA, USA) was utilized for all data analyses. Data are presented as mean±SE and were analyzed with 2-way analysis of variance (ANOVA) with Holm-Šidák's multiple comparisons test. The ROUT method with a maximum false discovery rate of 1% was applied to physiologic outcomes to detect outliers. Survival data were plotted as Kaplan-Meier curves and analyzed using the log-rank test. A two-tailed p<0.05 was considered significant.

### Example 4: Results

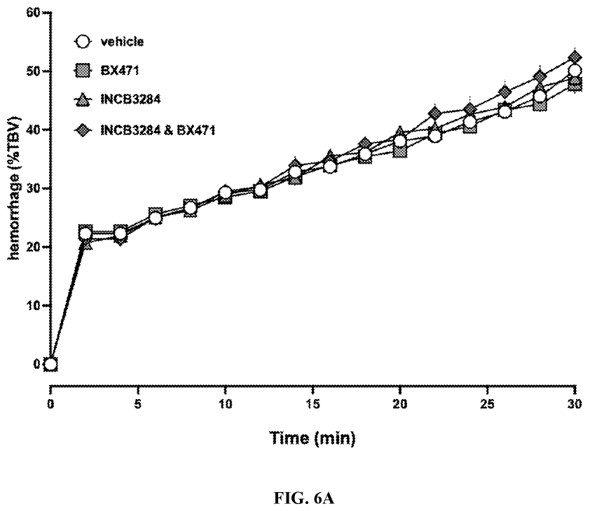

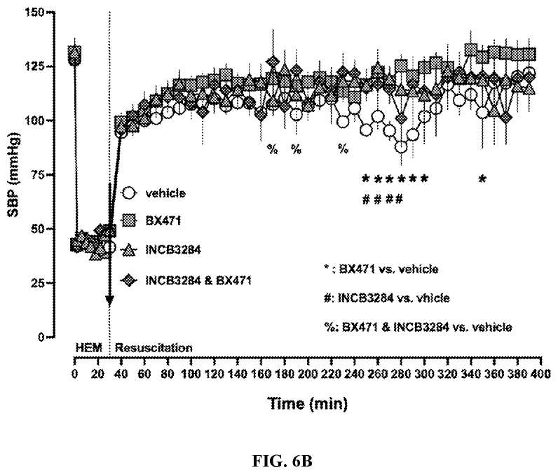

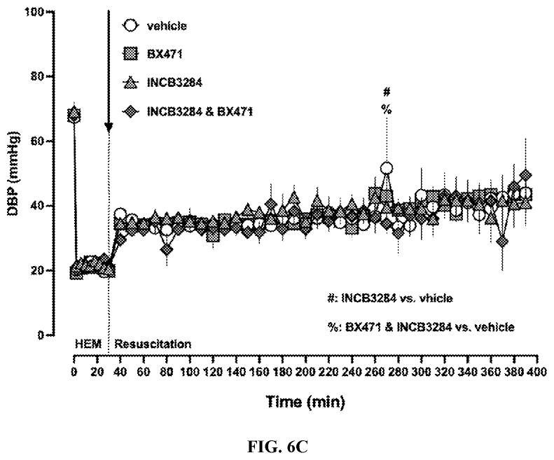

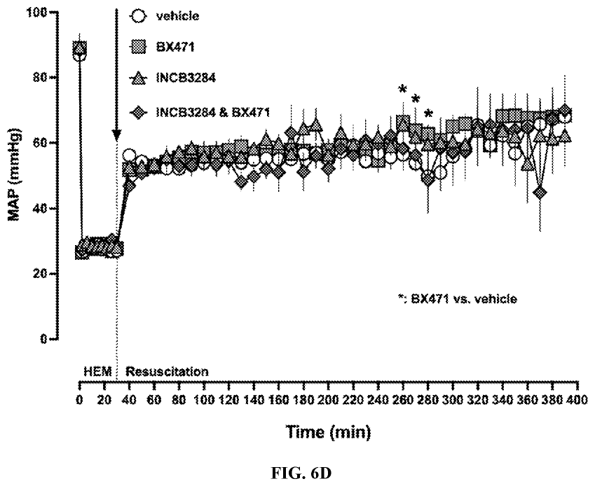

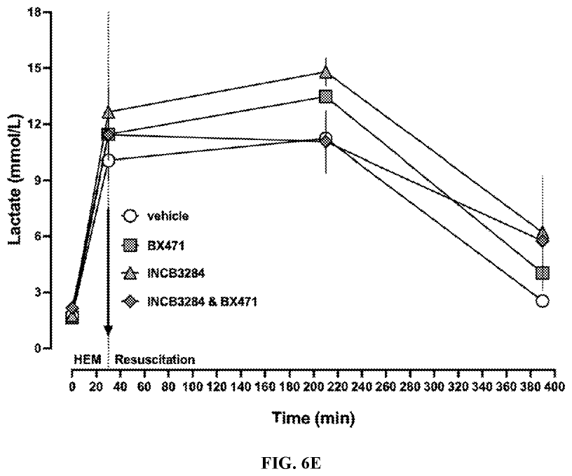

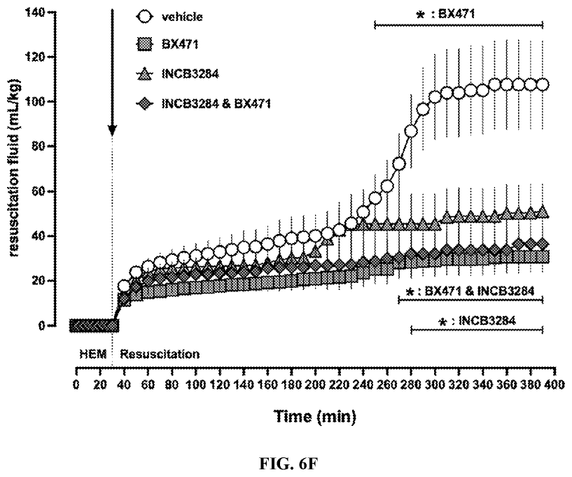

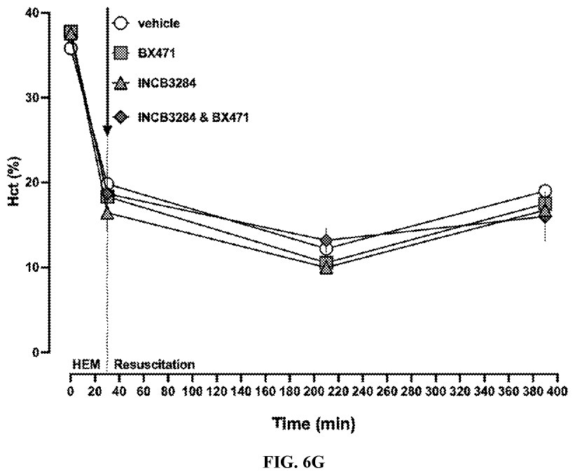

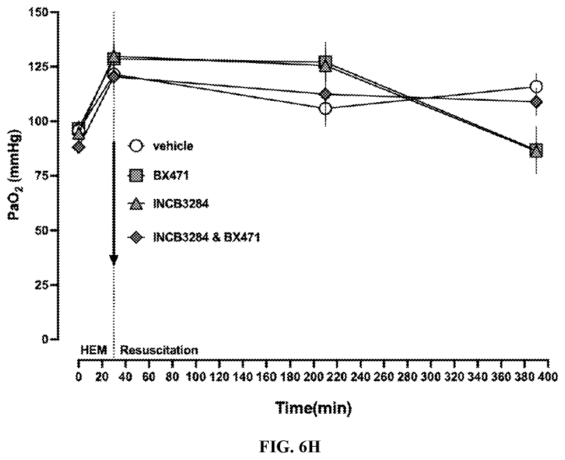

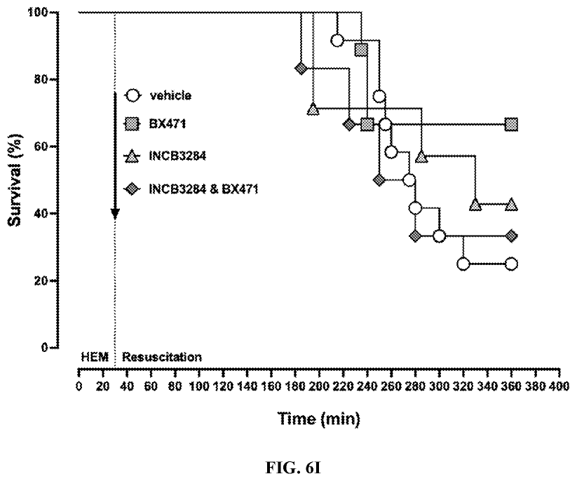

To evaluate and compare effects of BX471 and INCB3284 after hemorrhagic shock, animals were treated with a single intravenous injection of 1 mL of vehicle, 0.5 μmol/kg of BX471, 5 μmol/kg of INCB3284, or with a combination of 0.5 μmol/kg of BX471 plus 5 μmol/kg of INCB3284, at the beginning of fluid resuscitation. There were no differences in any of the physiological parameters among the groups at the beginning of the experiments. The hemorrhage volumes to maintain a MAP of 30 mmHg during the shock period were comparable among all groups (FIG. 6A). FIG. 6B-6D show the average SBP, diastolic blood pressure (DBP) and MAP, respectively, during the entire observation period. As compared with vehicle treated animals, systemic blood pressures of the animals in the treatment groups showed only short lasting and transient elevations throughout the resuscitation period. In all animals, blood lactate concentrations increased from baseline to 10-12.6 mmol/L at the end of the shock period and declined in surviving animals during subsequent fluid resuscitation (FIG. 6E). Blood lactate concentrations were indistinguishable among the groups (p>0.05 vs. vehicle for all treatment groups).

The cumulative resuscitation fluid requirements to maintain hemodynamics are shown in FIG. 6F. In vehicle-treated animals, cumulative fluid requirements to maintain hemodynamics sharply increased between t=220 min to t=300 min and averaged 108±20 mL/kg at the end of the observation period. Cumulative fluid requirements at the end of the observation period averaged 31±8 mL/kg in animals treated with BX471, and 51±12 mL/kg and 36.5±8 mL/kg in animals treated with INCB3284 and in animals treated with BX471 plus INCB3284, respectively (p<0.05 vs. vehicle treated animals for all treatment groups). The resuscitation fluid volume (mL) to hemorrhage volume (in mL of blood) ratios (R/H-ratios) averaged 3.5±0.6 in vehicle-treated animals, and 1.0±0.2, 1.6±0.4 and 1.1±0.2 after BX471, INCB3284 and BX471 plus INCB3284 treatment, respectively (p<0.05 vs. vehicle for all). There were no statistically significant differences in cumulative fluid requirements or R/H-ratios among the treatment groups. Hematocrit values (FIG. 6G) and PaO₂ (FIG. 6H) were comparable among all experimental groups. Mortality was 75% with vehicle treatment and 33%, 57% and 66% with BX471, INCB3284 and BX471 plus INCB3284 treatment, respectively (FIG. 6I, p>0.05 vs. vehicle treatment for all). The median survival time was 277.5 min with vehicle treatment, undefined with BX471 treatment, and 330 min and 265 min with INCB3284 and BX471 plus INCB3284 treatment, respectively (p>0.05 among all groups).

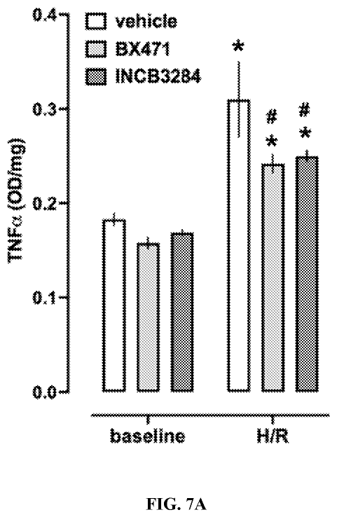

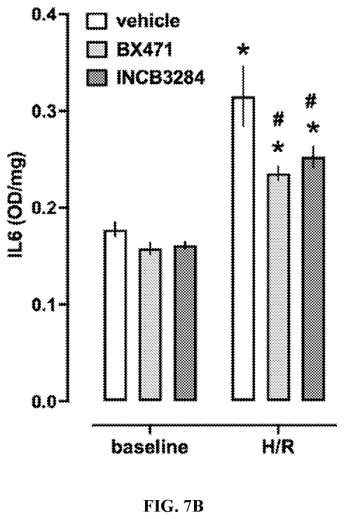

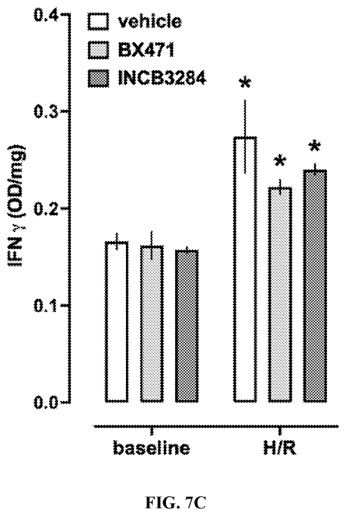

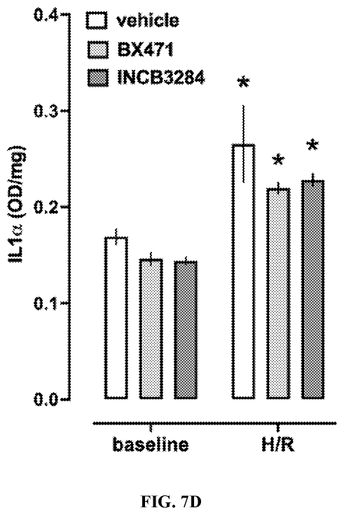

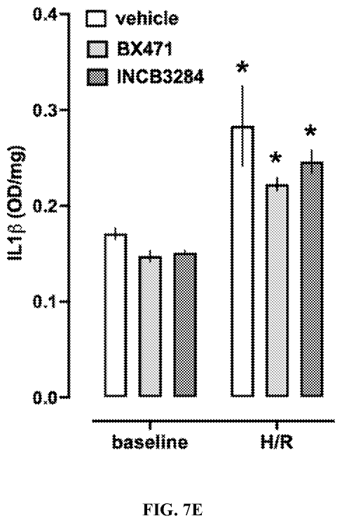

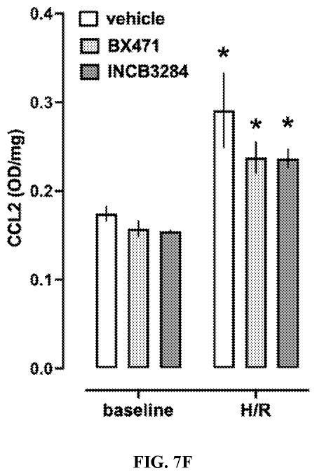

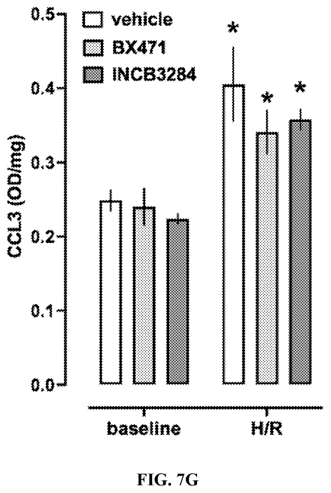

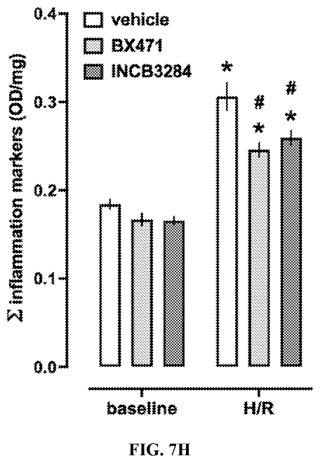

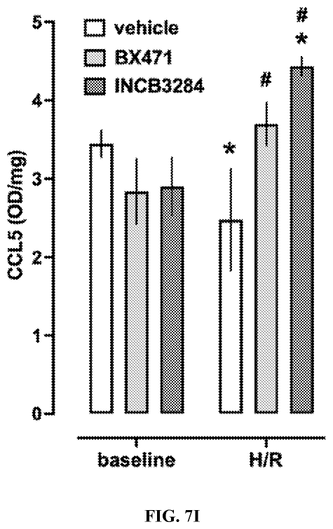

To assess whether BX471 or INCB3284 modulate the systemic inflammatory response to hemorrhage and fluid resuscitation, the concentrations of a panel of serum inflammation markers were measured at baseline and at the end of the experiments in animals that survived at least until t=330 min (FIGS. 7A-7I). Serum from animals after treatment with BX471 plus INCB3284 was not analyzed because two animals survived longer than t=330 min. Baseline concentrations per mg of total protein of the inflammation markers were comparable in vehicle-, BX471- and INCB3284-treated animals (p>0.05 vs vehicle treatment for all). As compared with baseline concentrations, serum levels of TNFα, IL6, IFNγ, IL1α, IL1β, CCL2 and CCL3 significantly increased during fluid resuscitation after hemorrhage in all animals (FIGS. 7A-7G). When compared with vehicle-treated animals, serum concentrations of TNFα and IL6 during resuscitation were significantly lower in BX471 and INCB3284 treated animals (FIGS. 7A-7B). While serum concentrations of IFNγ, IL1α, IL1β, CCL2 and CCL3 during resuscitation were also lower in BX471 and INCB3284 treated animals, as compared with vehicle-treated animals, these differences did not reach statistical significance. However, when all inflammation markers that increased during resuscitation were combined and compared among the groups, serum concentrations during resuscitation were significantly lower in BX471 and INCB3284 treated animals, as compared with vehicle treated animals (FIG. 7H). In contrast, serum concentrations of CCL5 decreased during resuscitation with vehicle treatment but increased with BX471 and INCB3284 treatment (FIG. 7I).

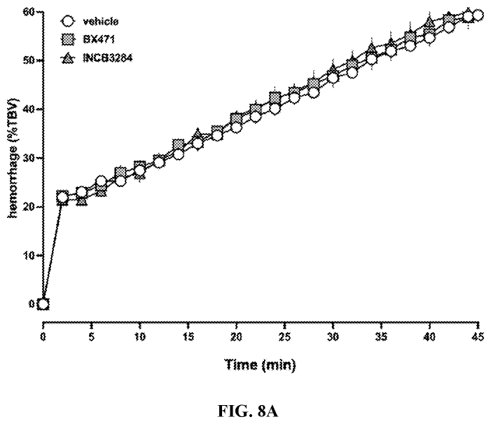

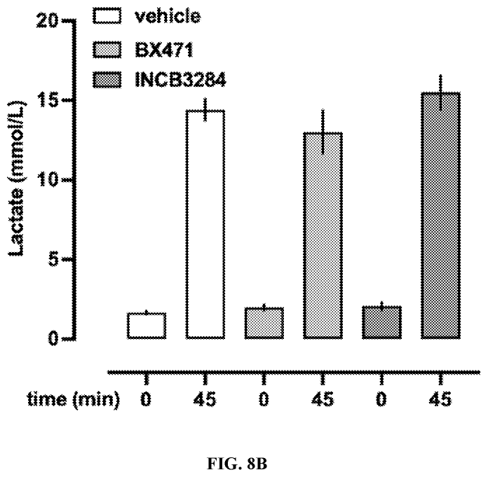

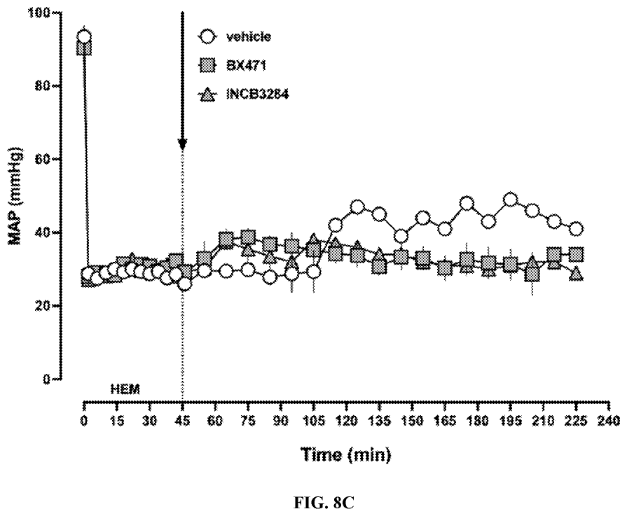

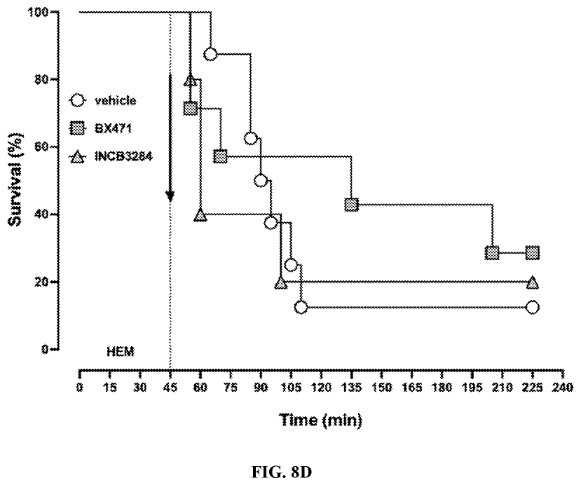

To evaluate and compare effects of BX471 and INCB3284 on shock tolerance in the absence of fluid resuscitation, animals were hemorrhaged to a MAP of 30 mmHg for 45 min, injected with 1 mL of vehicle, 0.5 μmol/kg of BX471 or 5 μmol/kg of INCB3284, and then observed until t=225 min without further intervention (FIGS. 8A-8D). Two animals died during the hemorrhage period before vehicle or drug injection. The hemorrhage volumes to maintain MAP at 30 mmHg for 45 min (FIG. 8A) and increases in blood lactate concentrations during the hemorrhage period (FIG. 8B) were comparable among all groups. As shown in FIG. 8C, MAP did not recover spontaneously during the observation period. With vehicle treatment, 1 out of 8 animals survived the observation period and the median survival time was 92.5 min (FIG. 8D). Two out of 7 animals treated with BX471 and 1 out of 5 animals treated with INCB3284 survived the observation period (FIG. 8D). The median survival times were 135 min with BX471 treatment and 60 min with INCB3284 treatment. Survival proportions and median survival times were not significantly different between the groups.

What is claimed is:

1. A resuscitation fluid, comprising a chemokine (C-C motif) receptor 1 (CCR1) inhibitor and an aqueous iso or hypertonic salt solution.

2. The resuscitation fluid of claim 1, further comprising a chemokine (C-C motif) receptor 2 (CCR2) inhibitor, a chemokine (C-C motif) receptor 3 (CCR3) inhibitor, a chemokine (C-C motif) receptor 5 (CCR5) inhibitor, or combinations thereof.

3. The resuscitation fluid of claim 1, wherein the CCR1 inhibitor comprises BX471, CCX354, BMS-817399, MLN3897, CP-481715, pharmaceutically acceptable salts thereof, or any combination thereof.

4. The resuscitation fluid of claim 1, wherein the CCR1 inhibitor is BX471 or a pharmaceutically acceptable salt thereof.

5. A method of fluid resuscitation of a patient in need thereof, comprising administering to the patient a resuscitation fluid and a therapeutically effective amount of a CCR1 inhibitor.

6. The method of claim 5, further comprising administering a therapeutically effective amount of a CCR2 inhibitor, a CCR3 inhibitor, a CCR5 inhibitor, or combinations thereof.

7. The method of claim 5, wherein the patient has hemorrhagic shock.

8. The method of claim 5, wherein the patient is undergoing cardiovascular, abdominal, or transplant surgery.

9. The method of claim 5, wherein the patient is treated for ischemia, hypovolemic shock, myocardial infarction, or stroke.

10. The method of claim 5, wherein the patient is treated for trauma, burn, sepsis, or shock.

11. The method of claim 5, wherein the CCR1 inhibitor comprises BX471, CCX354, BMS-817399, MLN3897, CP-481715, pharmaceutically acceptable salts thereof, or any combination thereof.

12. The method of claim 5, wherein the CCR1 inhibitor is BX471 or a pharmaceutically acceptable salt thereof.

13. The method of claim 5, wherein the CCR1 inhibitor is administered separately from the resuscitation fluid.

14. The method of claim 5, wherein the CCR1 inhibitor is a component of the resuscitation fluid.

15. The method of claim 6, wherein the therapeutically effective amount of the CCR2 inhibitor, the CCR3 inhibitor, the CCR5 inhibitor, or combinations thereof is from about 0.01 μmol/kg to 10 μmol/kg of the patient.

16. The method of claim 5, wherein the therapeutically effective amount of the CCR1 inhibitor is from about 0.01 μmol/kg to 10 μmol/kg of the patient.

17. The method of claim 5, wherein the therapeutically effective amount of the CCR1 inhibitor is from about 0.01 μmol/kg to 1 μmol/kg of the patient.

18. The method of claim 5, wherein the resuscitation fluid is lactated Ringer's solution.

19. The method of claim 5, wherein the resuscitation fluid is administered in an amount that is less than about three times the amount of fluid lost by the patient.

20. The method of claim 5, wherein the resuscitation fluid is administered in an amount that is from about the same to 1.5 times the amount of fluid lost by the patient.
